(19) United States
(12) Patent Application Publication
(10) Pub. No.: US 2021/0227823 A1
(43) Pub. Date: Jul. 29, 2021

(54) STABILIZED CHEMICAL COMPOSITION

(71) Applicant: Syngenta Crop Protection AG, Basel (CH)

(72) Inventors: Jeffrey David Fowler, Greensboro, NC (US); Sejong Kim, Greensboro, NC (US); Natalia Lebedeva, Greensboro, NC (US); Jelena Narsale, Greensboro, NC (US)

(73) Assignee: Syngenta Crop Protection AG, Basel (CH)

(21) Appl. No.: 17/054,388

(22) PCT Filed: May 10, 2019
(86) PCT No.: PCT/US2019/031644

§ 371 Date: Nov. 10, 2020

Related U.S. Application Data
(60) Provisional application No. 62/670,271, filed on May 11, 2018.

(51) Int. Cl. A01N 25/10; B01J 13/00; C08J 3/075; C05G 5/27; A01N 25/04; A01N 25/30; A01N 25/14; A01C 1/06

(52) U.S. Cl. CPC A01N 25/10; B01J 13/0065; C08J 3/075; A01C 1/06; A01N 25/04; A01N 25/30; A01N 25/14; C05G 5/27

(57) ABSTRACT

Stabilized liquid agrochemical compositions are provided that comprise flowable, liquid dispersion concentrates comprising a) a continuous liquid phase; and b) a dispersed phase comprising a dispersion of gel-like polymer matrix particles having a hardness greater than 0.01 MPa and less than 6 MPa, and where the outside surfaces of the particles comprise a colloidal solid material and the particles have a agrochemically active ingredient distributed therein The agrochemically active ingredient may be solid or liquid and is distributed within the polymer matrix particle. The compositions of the invention can be used directly or with dilution to combat pests or as plant growth regulators.

The present invention relates to stabilized, liquid, chemical compositions, the preparation of such compositions and a method of using such compositions, for example, to combat pests or as plant growth regulators.

### BACKGROUND OF THE INVENTION

Agriculturally active ingredients (agrochemicals) are often provided in the form of concentrates suitable for dilution with water. Many forms of agricultural concentrates are known and these consist of the active ingredient and a carrier, which can include various components. Water-based concentrates are obtained by dissolving, emulsifying and/or suspending agriculturally active materials in water. Due to the relatively complex supply chain for crop protection agents, such concentrate formulations can be stored for long periods and may be subjected during storage and shipping to extreme temperature variations, high-shear and repetitive vibration patterns. Such supply chain conditions can increase the likelihood of formulation failure such as, for example, flocculation, thickening and sedimentation.

In some cases it may be desirable to combine different agrochemicals in a single formulation taking advantage of the additive properties of each separate agrochemical and optionally an adjuvant or combination of adjuvants that provide optimum biological performance. For example, transportation and storage costs can be minimized by using a formulation in which the concentration of the active agrochemical(s) is as high as is practicable and in which any desired adjuvants are “built-in” to the formulation as opposed to being separately tank-mixed. The higher the concentration of the active agrochemical(s) however, the greater is the probability that the stability of the formulation may be compromised, or that one or more components may phase separate. In addition formulation failure can be more challenging to avoid when multiple active ingredients are present because of physical or chemical incompatibilities between these chemicals such as, for example, when one active ingredient is an acid, a base, an oily liquid, a hydrophobic crystalline solid or a hydrophilic crystalline solid and the other active ingredient(s) has or have different properties.

In addition, spray tank mixes can contain a variety of chemicals and adjuvants that may interact and change the effectiveness of one or more of the agrochemicals included therein. Incompatibility, poor water quality and insufficient tank agitation can lead to reduced effectiveness of sprays, phytotoxicity and can affect equipment performance.

Considering the variety of conditions and special situations under which agrochemical liquid concentrate formulation are stored, shipped and used around the world, there remains a need for improved liquid polymer dispersions comprising agrochemicals, including water-soluble, water-dispersible or water-sensitive agrochemicals, having a mean particle size of the dispersed particles of >1000 nm and which provide additional stability benefits under at least some of those conditions and situations. There is a further need for such formulations having high loading that are stable when diluted with water under a wide range of field conditions.

Once delivered to the end-user, the agrochemical formulation needs to perform as intended. Specifically, the formulation needs to contact the surface of the plant part on which it has been applied so that the active ingredient can be delivered to the plant part or pest. Ideally, the formulation will adhere so that it will not easily wash off from rain or other applications of water. In some instances, the formulation would be applied to a seed or to a plant propagule. For these cases, the formulation will need to adhere to the surface of the seed or plant propagule, so that it will not dust-off during handling and be present when the seed or propagule is planted. Therefore, it would be advantageous to provide a formulation which has excellent adherence to its target surface, such as the surface of foliage, seeds, or propagules.

Known technologies for producing polymeric particles or modifying the properties of polymeric particles include those such as coacervation, melt-cooling, solvent evaporation, grinding monolithic polymer blocks, interfacial polymerization, imbibing polymers such latex, and using mobile species to increase permeability of a polymer particle. However, these technologies all have shortcomings that the present technology seeks to overcome as described below.

Coascervation is a method to prepare a disperse phase in liquid suspension by inducing a species that is in solution in the liquid phase to precipitate on the surface of the disperse phase. Obvious limitations intrinsic to this method involve the difficulty in forming particles of uniform composition and size, because the mechanism of inducing precipitation must be matched to the mass transfer rate at which the precipitating species can encounter existing disperse phase particles. If the rate is too slow, the precipitating species will become supersaturated and simply form particles of that single species. Coacervation is generally not compatible with the present technology where the polymer particles are formed of several species (e.g., a one monomer and a plasticizer), coacervation does not allow for independent control of the different rates of precipitation and mass transfer of the different species, so the process is intrinsically unsuitable. In one embodiment, the present technology overcomes these limitations because the monomer and plasticizer are homogeneous throughout the disperse phase emulsion before the cross-linking reaction whereby the polymer matrix is formed.

Solvent evaporation involves forming a polymer solution in a volatile solvent, emulsifying that solution in an immiscible second solvent and then removing the volatile solvent to leave a dispersion of polymer particles. A practical shortcoming to the method is that the volatile solvent is either lost to the atmosphere or must be recovered—either option involving extra cost, and the dilute volatile solvent will typically be flammable and or hazardous.

It is known that homogeneous matrix particles can be prepared by grinding large blocks, however the present technology involves gel-like particles of plasticized polymer matrix. It is not needed, nor possible, to grind soft particles, as such grinding is not a feasible preparation method.

Interfacial polymerization occurs when a disperse phase of one monomer is present in a solution of a second monomer and the rate of reaction of the two monomers is sufficiently faster than mass transfer that they substantially react at a surface where the concentration of the second monomer essentially drops to zero. It is intrinsic to this process that the disperse phase is not homogeneous because the second monomer cannot diffuse into the center before it reacts, and this results in a disperse phase with a polymer shell around an essentially polymer-free liquid. Distortion of such polymer-encapsulated droplets can result in breakage and release of the contents. The present technology overcomes this shortcoming by achieving substantial homogeneity of the polymer matrix within the disperse phase, and as just described, this homogeneity is incompatible with the reaction kinetics that result in interfacial polymerization.

Preformed dispersions in water of polymer particles, i.e., a latex, are a conventional means of delivering film-forming polymers capable of adhering to a surface. It is known that latexes can imbibe an organic phase and so in principle might be used to adhere that organic phase, which might comprise an active ingredient, to a surface. One limitation to imbibing polymers such as latex, is that under stress conditions, whether by temperature cycling or by dilution into high electrolyte fertilizer, failure in the dispersion stability of an imbibed latex results in the polymer particles congealing into fused agglomerates that would cause catastrophic equipment blockages. Another limitation is that dried deposits of imbibed latexes are effectively sticky glue coatings that cannot be removed and would render equipment unusable. By contrast the formulations of the present technology are extremely stable while in aqueous dispersion. Dried films are not sticky and can be washed off as necessary.

It is known that the permeability of a polymer matrix particle can be increased by including mobile species that are capable of dissolving into a liquid in which the particles are placed, whereby the departure of the mobile species creates cavities or pores through which an active ingredient can diffuse. The present invention incorporates plasticizers within the polymer matrix substantially throughout the period during which they have utility as a result of their plasticity. A mobile species that dissolves out of the polymer matrix in order to create pores cannot serve as a plasticizer and as such the two functions, plasticizer and permeability agent, are incompatible as used herein. A plasticized polymer matrix which has low cross-link density generally does not present a barrier to diffusion.

Therefore a mobile species which diffuses from a polymer matrix in accordance with the present technology would not measurably increase its permeability, and as such it would not be possible to function as a permeability agent.

### SUMMARY OF THE INVENTION

The present technology is related to the design of gel emulsion formulations which contain soft, gel-like, ductile polymer matrix microparticles with a hardness of greater than 0.001 MPa and less than 6 MPa, and loaded with at least one agrochemical active ingredient (AI), and the use of these gel microparticles (GM) formulations for applications on plant parts, such as foliar applications, and for treatment of plant propagules, including seeds. In one embodiment, the present technology is related to dust-off reduction of seed treatment products. In another embodiment, the present technology is related to improvements in adhesion to plants, rainfastness for foliar applications, and reduction in dislodgeable foliar residues (DFR's) on sprayed crops. In another embodiment, the present technology relates to an agrochemical formulation that results in improved safety (e.g., reduction in phytotoxicity) to the crop while maintaining pesticidal efficacy to the target pest—such improvement includes applications to a seed or to a grown or growing plant.

Stabilized liquid agrochemical compositions are provided which comprise flowable, liquid dispersion concentrates comprising: a) a continuous aqueous liquid phase; b) at least one dispersed phase comprising GM having a mean particle size of at least 1 microns to at least 100 microns and a hardness greater than 0.001 MPa and less than 6 MPa, wherein the outside surfaces of the particles comprise a colloidal solid material and wherein the particles have at least one chemical agent distributed therein. The GM are prepared from either a curable or polymerizable resin or a solidifiable thermoplastic polymer.

In one embodiment, the colloidal solid material is present in the dispersed phase in an amount effective to stabilize the polymer resin in an emulsion state during the process which is used to prepare the dispersed phase. In other embodiments, the dispersed phase comprises polymer particles prepared by solidifying a thermoplastic polymeric resin, curing a thermoset resin or polymerizing a thermoplastic resin. In another embodiment, the chemical agent is a solid and is distributed within the dispersed phase, or is a liquid and is distributed within the dispersed phase. In a further embodiment the continuous liquid phase is water or is a mixture of water and either a water-miscible liquid or a water-soluble solid. In some embodiments, the continuous liquid phase is non-aqueous.

In some embodiments, the GM is prepared in the presence of a plasticizer to provide a GM that has a hardness greater than 0.001 MPa and less than 6 MPa. In some embodiments, the GM is prepared using an appropriate choice of the polymer composition (e.g., the polymer chemistry and/or cross-linking architecture) to provide a GM that has a hardness greater than 0.001 MPa and less than 6 MPa. The polymer network properties may be monitored, for example, with differential scanning calorimetry (DSC), nanoindentation, and/or rheological techniques. When the at least one chemical agent is an agrochemically active ingredient, the compositions of the invention can be used directly or with dilution to combat pests or as plant growth regulators.

In accordance with one embodiment of the invention, it has been found that liquid dispersion concentrates of agrochemically active ingredients in a liquid can be prepared by using polymerized, cured or solidified polymeric resin to entrap the agrochemically active ingredients in a polymer matrix when a colloidal solid is used to stabilize the polymer resin in an emulsion state during the curing reaction or solidification process. At least one agrochemically active ingredient can be distributed within the polymer matrix which is dispersed as particles within the continuous liquid phase. Other active ingredients may optionally be dispersed, dissolved, emulsified, microemulsified or suspended within the continuous phase.

The liquid dispersion concentrates of the invention have a usefully long period of protection for water-soluble, water-dispersible, water-sensitive and other agrochemicals such that the chemical and physical stability of the formulation is improved and which provides a practical utility in terms of storage, shipment and use. The dispersion concentrates of the present technology also conveniently allow the combination of multiple active ingredients in a single formulation, irrespective of whether they are liquids or solids, by incorporating them separately or together in GM that are mutually physically compatible.

The aqueous dispersion concentrates of the invention have utility also outside the agricultural field where there is need to prepare stable formulations and deliver chemical agents to a target site. For these purposes the agrochemicals may be replaced with other chemical agents as required. In the context of the present invention, chemical agents therefore include any catalyst, adjuvant, vaccine, genetic vector, drug, fragrance, flavor, enzyme, spore or other colony forming unit (CFU), dye, pigment, adhesive or other component where release of the chemical agent from the formulation is required. In addition the aqueous dispersion concentrates may be dried to prepare a powder or granular product as desired.

The polymerizable resins suitable for use in preparing the dispersed phase cured polymer matrix can be selected from monomers, oligomers or prepolymers which are polymerizable to either thermoset or thermoplastic polymer particles. In accordance with the invention, the disperse phase polymer matrix also can be formed by dissolving polymers in a volatile, water-immiscible solvent that also contains at least one agrochemical, stabilizing this solution in water as a Pickering emulsion using colloidal stabilizers, and then heating this emulsion to evaporate the volatile solvent and form a disperse phase of a thermoplastic polymer matrix. In addition, the disperse phase polymer matrix can be formed by dissolving or suspending at least one agrochemically active ingredient in a non-aqueous liquid mixture comprising a melt of at least one suitable thermoplastic polymer, emulsifying said dispersion concentrate into a heated aqueous liquid to a mean droplet size of 1-200 microns, which liquid also contains a colloidal solid as (Pickering) emulsion stabilizer; and cooling the emulsion to produce thermoplastic polymeric particles.

The present invention further relates to “gel” or “gel-like” polymer matrix particles comprising an entrapped agrochemical that is either homogeneously or non-homogeneously distributed within such particle or present in the form of domains within such particle and wherein the outside surface regions of the particles comprise a colloidal solid material. The term “gel” and “gel-like” as used herein is meant as non-limiting common descriptor and not to impart a definition or limitation of “gel” or “gel-like” on to the polymer particle.

The present invention also includes a method for combating or controlling pests or regulating the growth of plants at a locus such as soil or foliage which comprises treating said locus with a dispersion concentrate according to the invention or dispersing a concentrate according to the present invention in water or liquid fertilizer and treating said locus with the obtained diluted aqueous end-use formulation.

### BRIEF DESCRIPTION OF THE DRAWINGS

FIG. 1 is schematic representation of the gel particle with a clay colloidal solid in accordance with the present invention.

FIG. 2 is cross-section, schematic representation, of FIG. 1.

FIG. 3 is schematic representation of the gel particle with a substantially spherical colloidal solid in accordance with the present invention

FIG. 4 is cross-section, schematic representation, of FIG. 3.

FIG. 5 is cross-section, schematic representation of the gel particle with a clay colloidal solid and a solid active ingredient distributed with the polymeric matrix in accordance with the present invention.

FIG. 6 is a graph representing the data of Table 6a.

FIG. 7 is a graph representing the data of Table 6b.

FIG. 8 is a graph representing the data of Table 6c.

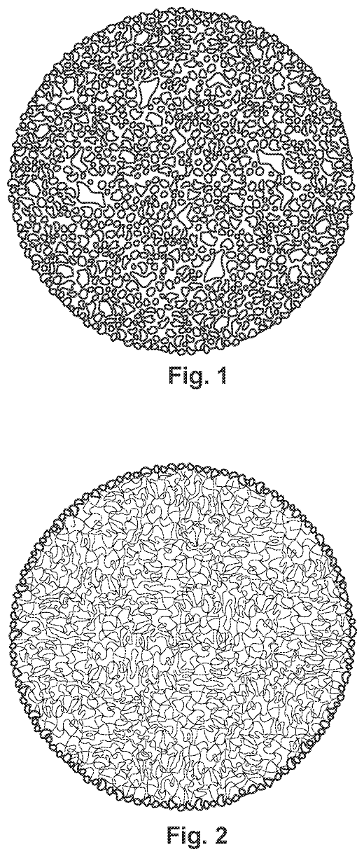

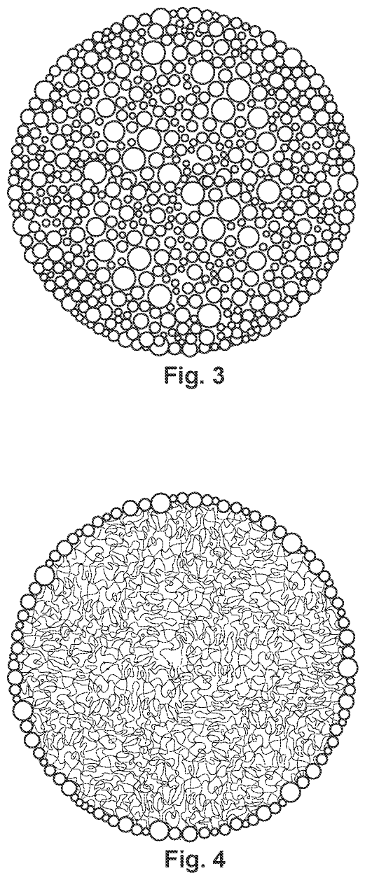

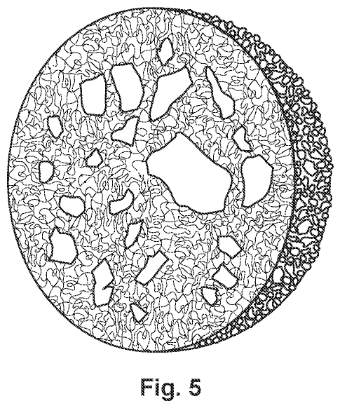

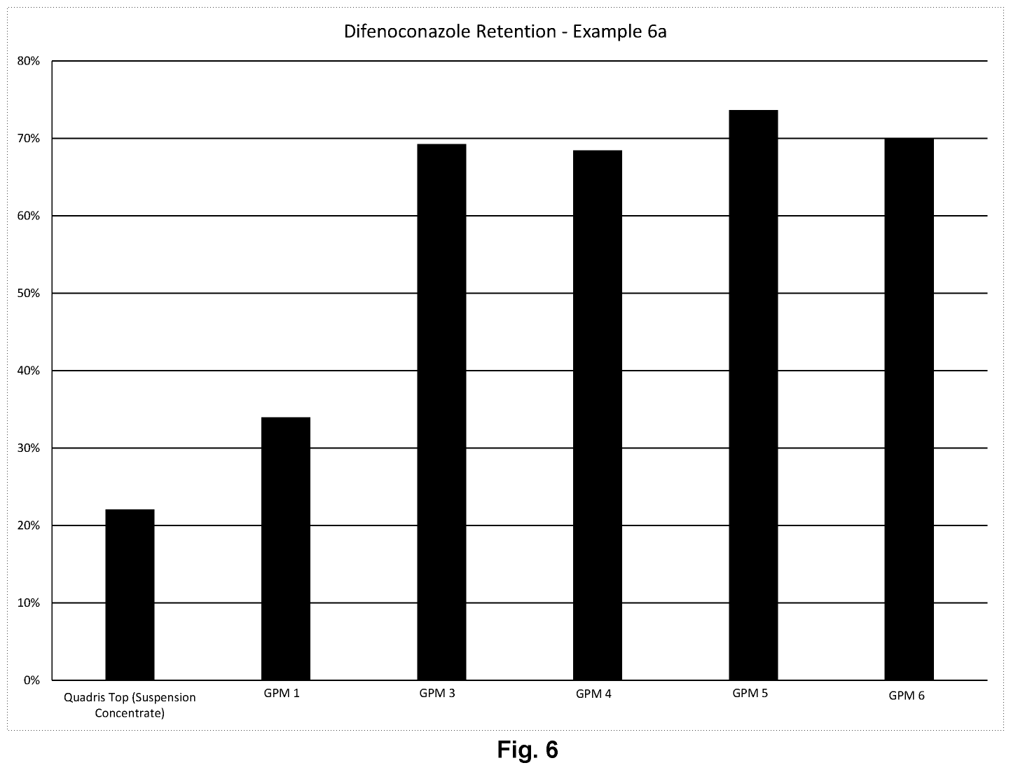

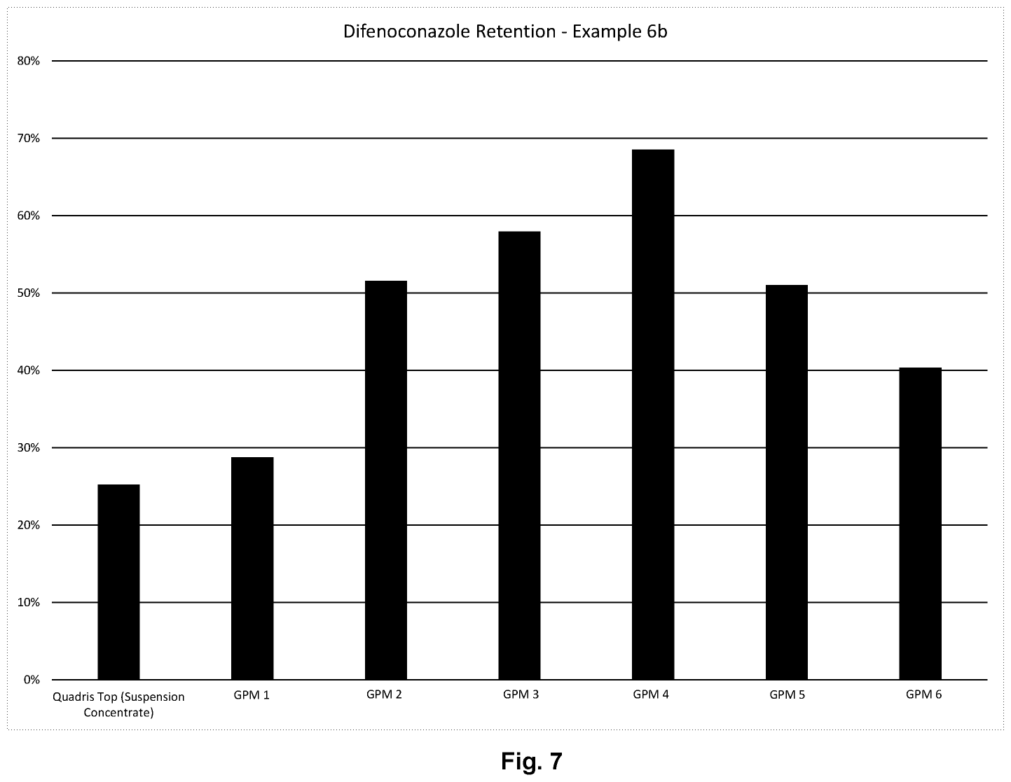

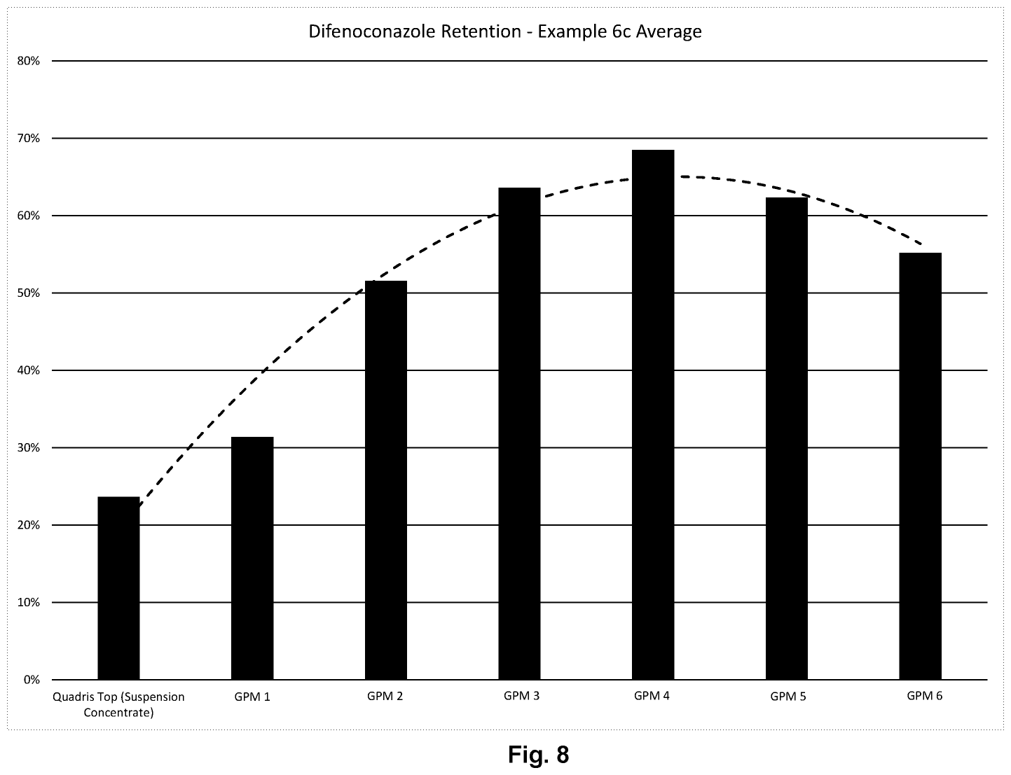

### DETAILED DESCRIPTION OF THE INVENTION

Accordingly, in one embodiment, the liquid dispersion concentrate compositions of the present invention comprise:
a) a continuous liquid phase, optionally comprising at least one chemical agent and optionally a polymeric dispersant; and
b) at least one dispersed phase comprising a polymer matrix microparticle, wherein the polymer matrix microparticle has a hardness greater than 0.001 MPa and less than 6 MPa, and wherein the outside surfaces of the particle comprise a colloidal solid material, and optionally comprises a plasticizer, and wherein the polymer particles have at least one chemical agent distributed therein.

In one embodiment, the chemical agents are agrochemically active ingredients.

In one embodiment, the colloidal solid material is a Pickering colloid emulsion stabilizer.

In one embodiment, the GM comprise an entrapped agrochemical that is either homogeneously on non-homogeneously distributed within such particles or present in the form of domains within such particles.

In the context of the present invention, mean particle or droplet size indicates the volume-weighted mean, commonly designated Dv50 as determined by dynamic light scattering.

In the context of the present invention, particle hardness is measured by the nanoindenter technique. The nanoindentation technique has been widely used to characterize the mechanical properties of materials at a surface. It is based on the following standards for instrumentation: ASTM E2546 and ISO 14577. Nanoindentation uses an established methodology where an indenter tip (typically conical for relatively soft samples) with a known geometry is driven into a specific site of the material, by applying an increasing normal load. Once a pre-set maximum value has been reached, the normal load is reduced until complete relaxation occurs. During the experiment, the position of the indenter relative to the sample surface is precisely monitored with a high precision capacitive sensor. The resulting load/displacement curves provide data specific to the mechanical nature of the material. Established physical models are used to calculate the hardness, the elastic modulus, and other mechanical properties of the material. The high spatial resolution of nanoindentation allows for tests of local mechanical properties.

In one embodiment, the agrochemically active ingredient is a solid and is distributed within the dispersed phase or is a liquid and is distributed within the dispersed phase.

In another embodiment, the dispersion concentrates for use in the liquid agrochemical compositions of the present invention are those that are formed using curing agents, monomers, oligomers, prepolymers or blends thereof that exhibit a slow curing or polymerization reaction when combined with the curing agents at ambient conditions. Particularly suitable are those curing agents, monomers, oligomers, prepolymers or blends thereof that exhibit no significant increase in viscosity under ambient conditions for a period of at least 15 minutes, more particularly 30 minutes, most particularly 1 hour, after mixing with the curing agent.

In accordance with one embodiment of the invention, polymerizable thermoset resins are understood to include all molecules that may be irreversibly polymerized or cured to form a polymeric matrix that does not melt or deform at elevated temperatures below the point of thermal decomposition. The polymerization reaction may be initiated thermally, by addition of chemical curing agents or by suitable irradiation to create radicals or ions such as by visible, UV, microwave or other electromagnetic irradiation, or electron beam irradiation. Examples include the phenolics, ureas, melamines, epoxies, polyesters, silicones, rubbers, polyisocyanates, polyamines and polyurethanes. In addition, bioplastic or biodegradable thermoset resins may be used including epoxy or polyester resins derived from natural materials such as vegetable oil, soy or wood and the like.

In accordance with another embodiment of the invention, polymerizable thermoplastic resins are understood to include all molecules that may be polymerized or cured to form a polymeric matrix that can melt or deform at elevated temperatures below the point of thermal decomposition. The polymerization reaction may be initiated thermally, by addition of chemical curing agents or by suitable irradiation to create radicals or ions such as by visible, UV or other electromagnetic irradiation, or electron beam irradiation. Examples of suitable ethylenically unsaturated monomers include styrene, vinyl acetate, a-methylstyrene, methyl methacrylate, those described in US 2008/0171658 and the like. Examples of thermoplastic polymers for polymer particles that can be prepared from in-situ mini-emulsion polymerization include polymethylmethacrylate, polystyrene, polystyrene-co-butadiene, polystyrene-co-acrylonitrile, polyacrylate, polyalkyl acrylate, polyalkyl acetate, polyacrylonitrile or their copolymers.

In accordance with yet another embodiment of the invention, solidifiable thermoplastic resins are understood to include all molecules that may be dissolved in a volatile solvent such that the solvent may be evaporated by heating to create a polymeric matrix that can melt or deform at elevated temperatures below the point of thermal decomposition. The volatile solvent is chosen to be immiscible with the continuous aqueous phase and sufficiently volatile that it can be conveniently removed from the composition by heating to a temperature below that where any significant decomposition occurs. Examples include polymers of the ethylenically unsaturated monomers described above, as well as polymers such as cellulose acetate, polyacrylates, polycaprolactone and polylactic acid. There may also be mentioned polymethylmethacrylate, polystyrene, polyethylvinyl acetate, cellulose acetate, polyacrylate, polyacrylonitrile, polyamide, polyalkyleneterephthalate, polycarbonate, polyester, polyphenylene oxide, polysulfone, polyimide, polyetherimide, polyurethane, polyvinylidene chloride, polyvinyl chloride, polypropylene and waxes, etc. In addition, bioplastic or biodegradable polymers such as thermoplastic starch, polylactic acid, polyhydroxy alkanoate, polycaprolactone, polyesteramide are also suitable for use in preparing polymer particles. Examples of volatile solvents include alkanes such as hexane and heptane, aromatic solvents such as benzene and toluene and halogenated solvents such as dicholoromethane and trichloromethane. Other examples of suitable polymers and solvents are described in WO2011/040956A1.

The term “polymer matrix particle” or “polymer matrix microparticle” as used herein means a polymer particle that is substantially uniform in density and polymer compositional make-up throughout the particle itself.

The term “microparticle” is a term that is generally used to describe particles that are microscopic in size. The polymer matrix particles of the present technology differ from microcapsules, which are composed of a distinct shell wall and hollow core. In accordance with the invention, the polymer matrix microparticles of the dispersed phase have a Dv50 particle size of from 1 to 200 microns, more particularly from 1 to 100 microns and most particularly, from 1 to 80 microns and 1-30 microns.

In one embodiment, suitable polymerizable resins and polymer solutions are those which are substantially immiscible with the liquid used in the continuous phase.

In the context of the present invention, a colloidal solid material is one whose properties of interest are determined by its surface interactions with other materials. Colloidal solids are therefore necessarily those with high specific surface area, typically above 10 m²/g. For example, colloidal solids are able to stabilize emulsions of immiscible liquids, as described for instance in WO 2008/030749. When serving for this purpose, such colloidal solids may be called Pickering colloids, colloidal emulsion stabilizers, or other equivalent terms. Functional tests are known for whether a colloidal solid can stabilize an emulsion as used herein. Not all colloidal solids are able to stabilize an emulsion of any given pair of immiscible liquids, and such a functional test may be used by those skilled in the art to identify a suitable colloid.

In another embodiment, where the continuous phase is aqueous, the affinity of the aqueous liquids suitable for use in the continuous phase a) for the agrochemically active ingredient distributed in the dispersed phase b) is such that substantially all of the agrochemically active ingredient remains in the dispersed solid phase and substantially none migrates to the continuous phase. Those skilled in the art will readily be able to determine whether a particular aqueous liquid meets this criterion for a specific agrochemically active ingredient in question by following any standard test procedure for determining the partition coefficient of a compound (in this case, the agrochemically active ingredient of the dispersed phase) between the continuous phase and the dispersed solid phase. Accordingly, the dispersed phase b) is immiscible with the continuous phase a).

In a further embodiment, the aqueous liquids suitable for use in the continuous phase a) are solutions of water-soluble solutes in water.

Water-soluble solutes suitable for use in the continuous phase include salts such as halides, nitrates, sulfates, carbonates, phosphates, nitrites, sulfites, nitrides and sulfides of ammonium and of metals such as those of groups 1 to 12 of the periodic table. Other suitable solutes include sugars and osmolytes such as polysaccharides, proteins, betaines and amino acids.

In one embodiment, the aqueous liquids suitable for use in the continuous phase a) are mixtures of water and a substantially water-miscible non-aqueous liquid. In the context of the invention, the term “substantially water-miscible” means a non-aqueous liquid that forms a single phase when present in water at a concentration up to at least 50 wt %.

Substantially water-miscible non-aqueous liquids suitable for use in the continuous phase a) include, for example, propylene carbonate; a water-miscible glycol selected from ethylene glycol, diethylene glycol, triethylene glycol, propylene glycol, dipropylene glycol, tripropylene glycol, butylene glycol, hexylene glycol and polyethylene glycols having a molecular weight of up to about 800; an acetylated glycol such as di(propylene glycol) methyl ether acetate or propylene glycol diacetate; triethyl phosphate; ethyl lactate; gamma-butyrolactone; a water-miscible alcohol such as propanol or tetrahydrofurfuryl alcohol; N-methyl pyrrolidone; dimethyl lactamide; and mixtures thereof. In one embodiment, the non-aqueous, substantially water-miscible liquid used in the continuous phase a) is a solvent for at least one optional agrochemically active ingredient.

In another embodiment, the aqueous, substantially water-miscible liquid used in the continuous phase a) is fully miscible with water in all proportions. Alternatively, the aqueous, substantially water-miscible liquid used in the continuous phase a) is a waxy solid such as polyethylene glycol having a molecular weight above about 1000 and the mixture of this waxy solid with water is maintained in the liquid state by forming the composition at an elevated temperature.

In another embodiment, the continuous liquid phase is a non-aqueous liquid. In another embodiment, the continuous liquid phase is a substantially water-immiscible, non-aqueous liquid. The water-immiscible, non-aqueous liquid may be selected from petroleum distillates, vegetable oils, silicone oils, methylated vegetable oils, refined paraffinic hydrocarbons, alkyl lactates, mineral oils, alkyl amides, alkyl acetates, and mixtures thereof.

In another embodiment, the continuous phase comprises a substantially water-miscible, non-aqueous liquid. The water-miscible, non-aqueous liquid may be selected from the group comprising propylene carbonate, ethylene glycol, diethylene glycol, triethylene glycol, propylene glycol, dipropylene glycol, tripropylene glycol, butylene glycol, hexylene glycol, polyethylene glycols having a molecular weight of up to about 800, di(propylene glycol) methyl ether acetate, propylene glycol diacetate, triethyl phosphate, ethyl lactate, gamma-butyrolactone, propanol, tetrahydrofurfuryl alcohol, N-methyl pyrrolidone, dimethyl lactamide, and mixtures thereof.

Those skilled in the art will appreciate that the quantities of water and the nature and quantity of the non-aqueous, water-miscible liquid or water-soluble solute can be varied to provide mixed aqueous liquids suitable for use in the continuous phase a) and these quantities can be determined without undue experimentation. In one embodiment, the aqueous continuous phase comprises 5 to 95 wt %, more preferably 30 to 90 wt %, ethylene glycol with the balance being water. In another embodiment, the aqueous continuous phase comprises 5 to 95 wt %, more preferably 30 to 90 wt %, glycerol with the balance being water.

In one embodiment, the liquid dispersion concentrate compositions of the present invention comprise a mixture of GM each containing one or more than one chemical agents (such as an agrochemically active ingredient). Each one of the chemical agent(s) is contained within the same or different dispersed phase GM, and each respective dispersed phase particle optionally includes a different polymer matrix as described above. Optionally each respective dispersed phase may have different particle sizes.

In one embodiment, the liquid dispersion concentrate compositions of the present invention comprise a dispersed phase in the form of finely divided, suspended polymer particles comprising a colloidal solid material at their outside surface and containing at least one agrochemically active ingredient.

The advantages of the liquid dispersion concentrate compositions (e.g. gel emulsions) of the present invention include: storage-stability for extended periods, multiple agrochemicals of different physical states may be conveniently combined in dispersions of mutually compatible particles; improved adhesion to surfaces where deposits are able to dry; reduced potential for crop injury due to the presence of solvents or other phytotoxic agents; improved acute toxicity; simple handling is made possible for users because dilution is made with water, or other liquid carrier, for preparation of application mixtures; the compositions can easily be resuspended or redispersed with only a minor amount of agitation and are not susceptible to coalescence when dilution is made with fertilizer solutions for preparation of application mixtures. The term “storage-stable” as used herein means that a given composition has a Dv50 that changes by less than about 20% over a period of 6 months at 70° F.

### Agrochemically Active Ingredients

The term “agrochemically active ingredient” refers to chemicals and biological compositions, such as those described herein, which are effective in killing, preventing, or controlling the growth of undesirable pests, such as, plants, insects, mice, microorganism, algae, fungi, bacteria, and the like (such as pesticidally active ingredients). The term may also apply to compounds that act as adjuvants to promote the uptake and delivery of other active compounds. The term may also apply to compounds that control the growth of plants in a desired fashion (e.g., plant growth regulators), to a compound which mimics the natural systemic activated resistance response found in plant species (e.g., plant activator) or to a compound that reduces the phytotoxic response to a herbicide (e.g., safener). If more than one is present, the agrochemically active ingredients are independently present in an amount that is biologically effective when the composition is diluted, if necessary, in a suitable volume of liquid carrier, e.g., water, and applied to the intended target, e.g., the foliage of a plant or locus thereof.

Examples of agrochemical active ingredients suitable for use within the continuous phase a) or disperse phase b) in accordance with the present invention include, but are not limited to: fungicides such as azoxystrobin, benzovindiflupyr, chlorothalonil, cyproconazole, cyprodinil, difenoconazole, fenpropidin, fludioxonil, mandipropamid, mefenoxam, paclobutrazole, picoxystrobin, propiconazole, pyraclostrobin, sedaxane, tebuconazole, thiabendazole and trifloxystrobin; herbicides such as acetochlor, alachlor, ametryn, anilofos, atrazine, azafenidin, benfluralin, benfuresate, bensulide, benzfendizone, benzofenap, bicyclopyrone, bromobutide, bromofenoxim, bromoxynil, butachlor, butafenacil, butamifos, butralin, butylate, cafenstrole, carbetamide, chloridazon, chlorpropham, chlorthal-dimethyl, chlorthiamid, cinidon-ethyl, cinmethylin, clomazone, clomeprop, cloransulam-methyl, cyanazine, cycloate, desmedipham, desmetryn, dichlobenil, diflufenican, dimepiperate, dimethachlor, dimethametryn, dimethenamid, dimethenamid-P, dinitramine, dinoterb, diphenamid, dithiopyr, EPTC, esprocarb, ethalfluralin, ethofumesate, etobenzanid, fenoxaprop-ethyl, fenoxaprop-P-ethyl, fentrazamide, flamprop-methyl, flamprop-M-isopropyl, fluazolate, fluchloralin, flufenacet, flumiclorac-pentyl, flumioxazin, fluorochloridone, flupoxam, flurenol, fluridone, flurtamone, fluthiacet-methyl, indanofan, isoxaben, isoxaflutole, lenacil, linuron, mefenacet, mesotrione, metamitron, metazachlor, methabenzthiazuron, methyldymron, metobenzuron, metolachlor, metosulam, metoxuron, metribuzin, molinate, naproanilide, napropamide, neburon, norflurazon, orbencarb, oryzalin, oxadiargyl, oxadiazon, oxyfluorfen, pebulate, pendimethalin, pentanochlor, pethoxamid, pentoxazone, phenmedipham, pinoxaden, piperophos, pretilachlor, prodiamine, profluazol, prometon, prometryn, propachlor, propanil, propazine, propham, propisochlor, propyzamide, prosulfocarb, pydiflumetofen, pyraflufen-ethyl, pyrazogyl, pyrazolynate, pyrazoxyfen, pyributicarb, pyridate, pyriminobac-methyl, quinclorac, siduron, simazine, simetryn, S-metolachlor, sulcotrione, sulfentrazone, tebutam, tebuthiuron, terbacil, terbumeton, terbuthylazine, terbutryn, thenylchlor, thiazopyr, thidiazimin, thiobencarb, tiocarbazil, triallate, trietazine, trifluralin, and vernolate; herbicide safeners such as benoxacor, dichlormid, fenchlorazole-ethyl, fenclorim, flurazole, fluxofenim, furilazole, isoxadifen-ethyl, mefenpyr; alkali metal, alkaline earth metal, sulfonium or ammonium cation of mefenpyr; mefenpyr-diethyl and oxabetrinil; insecticides such as abamectin, clothianidin, cyantraniliprole, cyanthraniliprole, emamectin benzoate, gamma cyhalothrin, imidacloprid, cyhalothrin and its enantiomers such as lambda cyhalothrin, tefluthrin, permethrin, resmethrin and thiamethoxam; nematicides such as fosthiazate, fenamiphos and aldicarb.

In one embodiment, the active ingredients in the continuous phase may be in the state of a solution, an emulsion, a microemulsion, a microcapsule or a particle or fine particle. In the context of the present invention, a fine particle is one substantially smaller than the dimensions of the GM of the dispersed phase, such that a plurality (at least 10) of active ingredient particles are within each particle of the dispersed phase, whereas a non-fine particle is one only slightly smaller than the dimensions of the GM of the dispersed phase, such that each polymeric particle contains only a few active ingredient particles.

Further aspects of the invention include a method of preventing or combating infestation of plant species by pests, and regulating plant growth by diluting an amount of concentrate composition with a suitable liquid carrier, such as water or liquid fertilizer, and applying to the plant, tree, animal or locus as desired. The formulations of the present invention may also be combined in a continuous flow apparatus with water in spray application equipment, such that no holding tank is required for the diluted product.

The liquid dispersion concentrate compositions can be stored conveniently in a container from which they are poured, or pumped, or into which a liquid carrier is added prior to application.

If a solid agrochemically active material is present, the solid active ingredient may be milled to the desired particle size prior to dispersion within the polymerizable resin (monomers, oligomers, and/or prepolymers, etc.) that will form the GM. The solid may be milled in a dry state using an air-mill or other suitable equipment as necessary, to achieve the desired particle size. The particle size may be a Dv50 particle size of about 0.2 to about 20 microns, suitably about 0.2 to about 15 microns, more suitably about 0.2 to about 10 microns.

As used herein, the term “agrochemically effective amount” means the amount of an agrochemical active compound which adversely controls or modifies target pests or regulates the growth of plants (PGR). For example, in the case of herbicides, a “herbicidally effective amount” is that amount of herbicide sufficient for controlling or modifying plant growth. Controlling or modifying effects include all deviation from natural development, for example, killing, retardation, leaf burn, albinism, dwarfing and the like. The term plants refers to all physical parts of a plant, including seeds, seedlings, saplings, roots, tubers, stems, stalks, foliage and fruits. In the case of fungicides, the term “fungicide” shall mean a material that kills or materially inhibits the growth, proliferation, division, reproduction, or spread of fungi. As used herein, the term “fungicidally effective amount” or “amount effective to control or reduce fungi” in relation to the fungicidal compound is that amount that will kill or materially inhibit the growth, proliferation, division, reproduction, or spread of a significant number of fungi.

As used herein, the terms “insecticide”, “nematicide” or “acaricide” shall mean a material that kills or materially inhibits the growth, proliferation, reproduction, or spread of insects, nematodes or acarids, respectively. An “effective amount” of the insecticide, nematicide or acaricide is that amount that will kill or materially inhibit the growth, proliferation, reproduction or spread of a significant number of insects, nematodes or acarids.

In one aspect, as used herein, “regulating (plant) growth”, “plant growth regulator”, PGR, “regulating” or “regulation” includes the following plant responses; inhibition of cell elongation, for example reduction in stem height and internodal distance, strengthening of the stem wall, thus increasing the resistance to lodging;

compact growth in ornamentals for the economic production of improved quality plants; promotion of better fruiting; increasing the number of ovaries with a view to stepping up yield; promotion of senescence of the formation of tissue enabling fruit to absciss; defoliation of nursery and ornamental bushes and trees for mail-order business in the fall; defoliation of trees to interrupt parasitic chains of infection; hastening of ripening, with a view to programming the harvest by reducing the harvest to one to two pickings and interrupting the food-chain for injurious insects.

In another aspect, “regulating (plant) growth”, “plant growth regulator”, “PGR”, “regulating” or “regulation” also includes the use of a composition as defined according to the present invention for increasing the yield and/or improving the vigor of an agricultural plant. According to one embodiment of the present invention, the inventive compositions are used for improved tolerance against stress factors such as fungi, bacteria, viruses and/or insects and stress factors such as heat stress, nutrient stress, cold stress, drought stress, UV stress and/or salt stress of an agricultural plant.

The selection of application rates relative to providing a desired level of pesticidal activity for a composition of the invention is routine for one of ordinary skill in the art. Application rates will depend on factors such as level of pest pressure, plant conditions, weather and growing conditions as well as the activity of the agrochemically active ingredients and any applicable label rate restrictions.

### Embodiments

The invention relates also to gel emulsion agrochemical compositions comprising
a) a continuous, aqueous liquid phase, optionally comprising at least one agrochemically active ingredient; and
b) at least one dispersed phase comprising polymer particles prepared from either a curable or polymerizable resin or a solidifiable thermoplastic polymer and comprising a colloidal solid material at their outside surface, wherein the hardness of the particles is greater than 0.001 MPa and less than 6 MPa, and wherein the particles have at least one agrochemically active ingredient distributed therein.

A further aspect of the invention relates to a dilute aqueous spray composition for combating pests or regulating the growth of plants at a locus comprising
a) a continuous aqueous phase comprising a suitable liquid carrier, such as water or a liquid fertilizer, in an amount sufficient to obtain the desired final concentration of each of the active ingredients in the spray composition;
b) at least one dispersed phase comprising polymer particles prepared from either a cureable or a polymerizable resin or a solidifiable thermoplastic polymer and comprising a colloidal solid material at their outside surface, wherein the hardness of the particles is greater than 0.001 MPa and less than 6 MPa, and wherein the particles have at least one agrochemically active ingredient distributed therein; and
c) optionally, at least one agrochemically active ingredient dispersed, dissolved, suspended, microemulsified and/or emulsified in the liquid carrier.

In another embodiment, the invention relates to a dilute pesticidal and/or PGR composition for ultra-low volume (ULV) application comprising:
a) a continuous phase comprising a carrier solvent having a flash point above 55° C. in an amount sufficient to obtain the desired final concentration of each of the active ingredients in the ULV composition;
b) at least one dispersed phase comprising polymer particles prepared from either a cureable or a polymerizable resin or a solidifiable thermoplastic and and comprising a colloidal solid material at their outside surface, wherein the hardness of the particles is greater than 0.001 MPa and less than 6 MPa and wherein the particles have at least one agrochemically active ingredient distributed therein.

The invention relates also to a method for combating or preventing pests in crops of useful plants or regulating the growth of such crops, said method comprising:
1) treating the desired area, such as plants, the plant parts or the locus thereof with a concentrate composition comprising:
a) a continuous aqueous liquid phase, optionally comprising at least one agrochemically active ingredient, and also optionally comprising at least one acidic or basic component;
b) at least one dispersed phase comprising polymer particles prepared from either a cureable or a polymerizable resin or a solidifiable thermoplastic and comprising a colloidal solid material at their outside surface, wherein the hardness of the particles is greater than 0.001 MPa and less than 6 MPa and wherein the particles have at least one agrochemically active ingredient distributed therein; or
2) diluting the concentrate composition, if necessary, in a suitable carrier, such as water, liquid fertilizer or a carrier solvent having a flash point above 55° C., in an amount sufficient to obtain the desired final concentration of each of the agrochemically active ingredients; and then treating the desired area, such as plants, the plant parts or the locus thereof with the dilute spray or ULV composition.

The term plants refers to all physical parts of a plant, including seeds, seedlings, saplings, roots, tubers, stems, flowers, stalks, foliage and fruits. The term locus refers to where the plant is growing or is expected to grow.

The composition according to the invention is suitable for all methods of application conventionally used in agriculture, e.g. pre-emergence application, post-emergence application, post-harvest and seed dressing. The compositions according to the invention are suitable for pre- or post-emergence applications to crop areas.

The compositions according to the invention are also suitable for combating and/or preventing pests in crops of useful plants or for regulating the growth of such plants. In some embodiments, the compositions may be applied by any method that is conventionally used, including spraying, dripping, and wicking. One advantage of the GM of the present formulations is that their small size permits an even coverage of plant stems and leaves where the distance between particles of the formulation is small. Thus, the formulation is more effective in contacting pests that damage the plant.

Preferred crops of useful plants include canola, cereals such as maize, barley, oats, rye and wheat, cotton, soya, sugar beets, fruits, berries, nuts, vegetables, flowers, trees, shrubs and turf. The components used in the composition of the invention can be applied in a variety of ways known to those skilled in the art, at various concentrations. The rate at which the compositions are applied will depend upon the particular type of pests to be controlled, the degree of control required, and the timing and method of application.

Crops are to be understood as also including those crops which have been rendered tolerant to herbicides or classes of herbicides (e.g. ALS-, GS-, EPSPS-, PPO-, ACCase and HPPD-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood to include those which are obtained by conventional methods of breeding or genetic engineering and contain so-called output traits (e.g. improved storage stability, higher nutritional value and improved flavour).

Other useful plants include turf grass for example in golf-courses, lawns, parks and roadsides, or grown commercially for sod, and ornamental plants such as flowers or bushes.

Crop areas are areas of land on which the cultivated plants are already growing or in which the seeds of those cultivated plants have been sown, and also areas of land on which it is intended to grow those cultivated plants.

Other active ingredients such as herbicide, plant growth regulator, algaecide, fungicide, bactericide, viricide, insecticide, acaricide, nematicide or molluscicide may be present in the formulations of the present invention or may be added as a tank-mix partner with the formulations.

The compositions of the invention may further comprise other inert additives.

Such additives include thickeners, flow enhancers, dispersants, emulsifiers, wetting agents, antifoaming agents, biocides, lubricants, fillers, drift control agents, deposition enhancers, adjuvants, evaporation retardants, freeze protecting agents, insect attracting odor agents, UV protecting agents, fragrances, and the like. The thickener may be a compound that is soluble or able to swell in water, such as, for example, polysaccharides of xanthans (e.g., anionic heteropolysaccharides such as RHODOPOL® 23 (Xanthan Gum)(Rhodia, Cranbury, N.J.)), alginates, guars or celluloses; synthetic macromolecules, such as modified cellulose-based polymers, polycarboxylates, bentonites, montmorillonites, hectonites, or attapulgites. The freeze protecting agent may be, for example, ethylene glycol, propylene glycol, glycerol, diethylene glycol, saccharose, water-soluble salts such as sodium chloride, sorbitol, triethylene glycol, tetraethylene glycol, urea, or mixtures thereof. Representative anti-foam agents are silicone oils, polydialkylsiloxanes, in particular polydimethylsiloxanes, fluoroaliphatic esters or perfluoroalkylphosphonic/perfluoroalkylphosphonic acids or the salts thereof and mixtures thereof. Suitable antifoams are polydimethylsiloxanes, such as Dow Corning® Antifoam A, Antifoam B or Antifoam MSA. Representative biocides include 1,2-benzisothiazolin-3-one, available as PROXEL® GXL (Arch Chemicals). Conventional surfactants may only be present at low concentrations because of their ability to form micelles in the aqueous phase, because these micelles extract solvent, plasticizer and/or active ingredient from the GM. Thus although conventional surfactants are useful to control the viscosity of dispersions of GM, at higher concentrations they have the potential to extract components from the particles and obviate their advantages. Therefore, compositions of the present technology may not contain conventional surfactants at concentrations above that at which they form micelles, which concentration is termed the critical micelle concentration (CMC). For this reason non-micellar polymeric dispersants are preferred to control the viscosity of dispersions of GM. Examples of conventional surfactants that form micelles are linear and branched alcohol ethoxylates and their acid esters, tristyryl-phenol ethoxylates and their acid esters, alkyl-phenol ethoxylates and their acid esters, linear or branched alkyl-aryl sulfonates such as dodecyl-benzene sulfonate, fatty acid ethoxylates, alkyl amine ethoxylates, block copolymers of ethylene oxide and higher alkylene (propylene-, butylene-) oxides. Examples of non-micellar polymeric dispersants include polyvinylpyrrolidone homopolymer with a molecular weight between 15-120 kDa, polyvinylpyrrolidone-vinyl acetate random copolymer, lignosulfonates, sulfonated urea-formaldehyde condensates, styrene acrylic copolymers, comb polymers with an alkyl backbone and side chains of polyacrylic acid, alkylated polyvinylpyrrolidone, and other general, non-emulsifying dispersants.

Dispersants are well known in the art and selection of such will have various factors dependent on a given formulation. Preferred dispersants, as noted above, include, without limitation, polyvinylpyrrolidone homopolymer with a molecular weight between 15-120 kDa, polyvinylpyrrolidone-vinyl acetate random copolymer, lignosulfonates, sulfonated urea-formaldehyde condensates, styrene acrylic copolymers, comb polymers with alkyl backbone and side chains of polyacrylic acid, alkylated polyvinylpyrrolidone, and other general, non-emulsifying dispersants.

The compositions of the invention may be mixed with fertilizers and still maintain their stability.

The compositions of the invention may be used in conventional agricultural methods. For example, the compositions of the invention may be mixed with water and/or fertilizers and may be applied preemergence and/or postemergence to a desired locus by any means, such as airplane spray tanks, irrigation equipment, direct injection spray equipment, knapsack spray tanks, cattle dipping vats, farm equipment used in ground spraying (e.g., boom sprayers, hand sprayers), and the like. The desired locus may be soil, plants, and the like.

The present technology further includes a method for treating seeds or plant propagules, comprising contacting said seeds or plant propagules with a composition of the present invention. The present technology can be applied to a seed or plant propagule in any physiological state, at any time between harvest of the seed and sowing of the seed; during or after sowing; and/or after sprouting. It is preferred that the seed or plant propagule be in a sufficiently durable state that it incurs no or minimal damage, including physical damage or biological damage, during the treatment process. A formulation may be applied to the seeds or plant propagules using conventional coating or pelleting techniques and machines, such as: fluidized bed techniques, the roller mill method, rotostatic seed treaters, and drum coaters. The seeds or plant propagules may be pre-sized before coating. After coating, the seeds or plant propagules are typically dried and then transferred to a sizing machine for sizing. Such procedures are known in the art. In some embodiments, a composition of the present invention is applied as one ingredient of a seed or plant propagule coating. The treated seeds may also be enveloped with a film over-coating to protect the coating. Such over-coatings are known in the art and may be applied using conventional fluidized bed and drum film coating techniques, for example.

Within the scope of the present invention are different methods of producing dispersed phase GM containing chemical agents, which are described in a manner wherein the chemical agents are agriculturally active ingredients. Each method results in a dispersed phase that comprise a GM having a hardness of the particles greater than 0.001 MPa and less than 6 MPa with at least one agriculturally active ingredient distributed therein, and a colloidal solid material at the surface of the particle.

The first method comprises the following steps:
1. preparing a dispersion concentrate by dissolving or suspending at least one agrochemically active ingredient in a non-aqueous curable liquid mixture comprising at least one suitable cross-linkable resin (comprising monomers, oligomers, prepolymers or blends thereof), optionally where the resin contains hydrophilic groups, optionally a suitable hardener, catalyst, plasticizer or initiator,
2. emulsifying said dispersion concentrate in to an aqueous liquid to a mean droplet size of 1-200 microns, where the liquid contains a colloidal solid as an emulsion stabilizer, optionally contains a plasticizer, and, optionally, certain suitable hardener, catalyst or initiator capable of diffusing into the dispersed uncured resin droplets; and
3. effecting crosslinking or cure of the cross-linkable resin mixture, and optionally thereafter imbibing a plasticizer, to produce cured thermoset polymeric particles having a hardness of the particles is greater than 0.001 MPa and less than 6 MPa with at least one agriculturally active ingredient distributed therein, and a colloidal solid material at the surface of the particle.

The second method is substantially identical to the first, except that the dispersion concentrate comprises as non-aqueous liquid a polymerizable resin instead of a cross-linkable resin. Instead of a curing reaction in step 3, the dispersed phase particles are formed by a polymerization reaction, so that the resulting dispersed phase comprises thermoplastic polymeric particles rather than thermoset polymeric particles.

The third method comprises the following steps:
1. dissolving or suspending at least one agrochemically active ingredient in a non-aqueous liquid mixture comprising at least one suitable solidifiable polymer dissolved in a volatile solvent, and one or more optional plasticizers;
2. emulsifying said solution in to an aqueous liquid to a mean droplet size of 1-200 microns, where the liquid contains a colloidal solid as an emulsion stabilizer and optionally contains a plasticizer; and
3. effecting evaporation of the volatile solvent by heating the emulsion to a temperature of about 30-120° C. for about 0.1-10 hr, and optionally thereafter imbibing a plasticizer, to produce thermoplastic polymer particles having a hardness greater than 0.001 MPa and less than 6 MPa with at least one agriculturally active ingredient distributed therein, and a colloidal solid material at the surface of the particle.

The fourth method of preparation comprises the following steps:
1. preparing a dispersion concentrate by dissolving or suspending at least one agrochemically active ingredient in a non-aqueous curable liquid mixture comprising a melt of at least one suitable solidifiable thermoplastic polymer and optionally an plasticizer;
2. emulsifying said dispersion concentrate in to a heated aqueous liquid to a mean droplet size of 1-200 microns, which liquid contains a colloidal solid as an emulsion stabilizer and optionally contains a plasticizer; and
3. cooling the emulsion, and optionally thereafter imbibing a plasticizer, to produce thermoplastic polymeric particles having a hardness greater than 0.001 MPa and less than 6 MPa with at least one agriculturally active ingredient distributed therein, and a colloidal solid material at the surface of the particle.

In situations where the active ingredient is soluble or miscible with the plasticizer, the four variant methods above may each be modified so that an active ingredient is added after the step of curing, solidifying or extracting solvent from the liquid emulsion droplets, so that the active ingredient is imbibed or dissolved into the GM's after formation rather than being present in the dispersion concentrate initially.

In one embodiment, the dispersion concentrate is prepared by:

a. dissolving or suspending at least one agrochemically active ingredient in a non-aqueous liquid mixture (premix) comprising at least one suitable curable or polymerizable resin (comprising monomers, oligomers, prepolymers or blends thereof), optionally a suitable hardener, plasticizer, catalyst or initiator;
b. emulsifying said solution or suspension in to an aqueous liquid to a mean droplet size of 1-200 microns, which liquid also contains a colloidal solid as an emulsion stabilizer and optionally contains a plasticizer, certain suitable hardener, catalyst or initiators capable of diffusing into the dispersed uncured or unpolymerized resin droplets; and
c. effecting crosslinking, cure or polymerization of the resin mixture, and optionally thereafter imbibing a plasticizer, to produce cured thermoset or polymerized thermoplastic resin polymer particles having a hardness greater than 0.001 MPa and less than 6 MPa with at least one agriculturally active ingredient distributed therein and a colloidal solid material at the surface of the particle, and which after curing are dispersed in the aqueous liquid.

In one embodiment, the dispersion concentrate is prepared by adding the hardener through the continuous phase, after the Pickering emulsion is formed, so that the dispersed phase premix is incapable of curing. Alternatively a first very slow-reacting hardener can be used in the dispersion concentrate, and then a second fast-curing hardener, an accelerator or catalyst can be added through the continuous phase. These second agents are added to the continuous phase after the dispersed phase is emulsified, so they must be chosen to be miscible in the continuous phase. Suitable fast cure water-miscible hardeners include diethylene triamine, triethylene tetramine, xylene diamine, polyethylene glycol diamine, isophorone diamine and polyoxypropylene diamine. Mixtures of hardeners may also be employed for extra flexibility.

In one embodiment, the dispersion concentrate is prepared by adding a premix of the dispersed phase to a premix of the continuous phase, wherein:
1) the premix of the dispersed phase is prepared by blending with a high shear mixer: at least one agriculturally active ingredient, at least one suitable curable or polymerizable resin monomer, oligomer, prepolymer or blend thereof, a suitable hardener, catalyst or initiator;
2) the premix of the continuous phase is prepared by blending with low shear mixer: an aqueous liquid with a colloidal solid as an emulsion stabilizer.

The resulting mixtures of the dispersed phase premix and the continuous phase premix are stirred under high shear conditions for a suitable time to form a Pickering emulsion and then heated or exposed to light or other electromagnetic radiation conditions (UV, microwave), as needed, in order to polymerize the dispersed phase. The shear rate and duration of the emulsification may be readily determined by one skilled in the art, guided by the following observations: if the shear rate is too low, the emulsion and resulting polymer matrix particles are relatively coarse and may be larger than desired; if the shear rate is instead too high or of too long a duration, the emulsion stabilizing colloid eventually becomes so depleted from the continuous phase that any new interfacial surface between the dispersed and continuous phases is effectively unprotected, at which point rapid coalescence or heteroflocculation of the dispersed phase occurs and the Pickering emulsion becomes inhomogeneous.

In one embodiment, the mixture of the dispersed phase premix and the continuous phase premix is stirred under high shear conditions for 5-10 min and heated to a temperature of about 30-120° C. for about 0.1-10 hr in order to effect the curing reaction.

In one embodiment, the dispersion concentrate is prepared by:
a. dissolving or suspending at least one agrochemically active ingredient in a non-aqueous liquid mixture comprising at least one suitable polymer dissolved in a volatile solvent;
b. emulsifying said solution in to an aqueous liquid to a mean droplet size of 1-200 microns, which liquid also contains a colloidal solid as (Pickering) emulsion stabilizer; and
c. effecting evaporation of the volatile solvent by heating the emulsion to a temperature of about 30-120° C. for about 0.1-10 hr to produce thermoplastic particles having a hardness greater than 0.001 MPa and less than 6 MPa with at least one agriculturally active ingredient distributed therein and a colloidal solid material at the surface of the particle, and which are dispersed in the aqueous liquid. If necessary more liquid may be added to the continuous phase to replace any liquid lost during the evaporation process.

Preferred polymerizable resins for use in preparing the polymer particles of the dispersed phase include thermosets such as epoxy resins, phenolic resins, aminoplast resins, polyester resins, polyacrylate, biodegradable polymer, polyurethane, and polyurea. Epoxy resins are particularly preferred. Combinations of these resins may also be used to achieve miscibility with the other components of the disperse phase and to control the polymerization kinetics.

Other suitable polymerizable resins for use in preparing the polymer particles of the dispersed phase include thermoplastics resins such as styrenes, methyl methacrylates, and acrylics. Combinations of these resins may also be used to achieve miscibility with the other components of the disperse phase.

Preferred thermoplastic polymers include polymers of the thermoplastic resins described above, as well as polymers such as cellulose acetate, polyacrylates, polycaprolactone and polylactic acid.

The polymerization reaction may be initiated thermally, by addition of chemical curing agents and/or catalysts or by suitable irradiation such as by visible, UV, microwave or other electromagnetic irradiation, electron beam irradiation, or ultrasonication to produce reactive species such as radicals or ions.

Suitable monomers for the present invention comprise vinylaromatic monomers, such as styrene, α-methylstyrene, divinylbenzene and the like, esters of α,β-monoethylenically unsaturated mono- and dicarboxylic acids, in particular the esters of acrylic acid, such as ethyl acrylate, n-butyl acrylate, trimethylolpropane triacrylate, pentaerythritol triacrylate and the esters of methacrylic acid, such as ethyl methacrylate, n-butyl methacrylate, n-hexyl methacrylate and the like. Suitable monomers are furthermore vinyl esters and allyl esters of aliphatic carboxylic acids, for example vinyl acetate and vinyl propionate, vinyl halides, such as vinyl chloride and vinylidene chloride, conjugated diolefins, such as butadiene and isoprene. Examples of suitable unsaturated monomers also include acrylamide, methacrylamide, acrylonitrile, methacrylonitrile, N-vinylformamide and N-vinylpyrrolidone, and also acrylic acid, methacrylic acid, styrenesulfonic acid, and vinylphosphonic acid.

Additional examples of polymers suitable for use in preparing the GM of the present invention include the phenolics, ureas, melamines, epoxies, silicones, polyisocyanates, polyamines and polyurethanes, polycarbonate, polyalkyleneterephthalate, polyphenylene oxide, polysulfone, polyimide, polyetherimide, polyhydroxy alkanoate, polycaprolactone, polyesteramide, and polylactic acid. In addition, biopolymer or biodegradable resins may be used derived from natural materials such as plants, algae, microbes or animals, including vegetable or algal oils, lignin, humic acid, glycoproteins, proteins, polypeptides, polysaccharides, cellulose or hemicellulose, and the like.

With respect to the epoxies, all customary mono-, di-, and polyepoxide monomers, prepolymers or blends thereof are suitable epoxy resins for the practice of this invention. In one embodiment, suitable epoxy resins are those that are liquid at ambient temperature. The di- and polyepoxides may be aliphatic, cycloaliphatic or aromatic compounds. Typical examples of such compounds are the diglycidyl ethers of bisphenol

A, glycerol or resorcinol, the glycidyl ethers and β-methylglycidyl ethers of aliphatic or cycloaliphatic diols or polyols, including those of hydrogenated bisphenol A, ethylene glycol, 1,2-propanediol, 1,3-propanediol, 1,4-butanediol, diethylene glycol, polyethylene glycol, polypropylene glycol, glycerol, trimethylolpropane or 1,4-dimethylolcyclohexane or of 2,2-bis(4-hydroxycyclohexyl)propane, the glycidyl ethers of di- and polyphenols, typically resorcinol, 4,4′-dihydroxydiphenylmethane, 4,4′-dihydroxydiphenyl-2,2-propane, novolaks and 1,1,2,2-tetrakis(4-hydroxyphenyl)ethane, Further examples are N-glycidyl compounds, including diglycidyl compounds of ethylene urea, 1,3-propylene urea or 5-dimethylhydantoin or of 4,4′-methylene-5,5′-tetramethyldihydantoin, or those such as triglycidyl isocyanurate, or biodegradable/bio-derived epoxies (vegetable oil-based).

Further glycidyl compounds of technical importance are the glycidyl esters of carboxylic acids, especially di-and polycarboxylic acids. Typical examples are the glycidyl esters of succinic acid, adipic acid, azelaic acid, sebacic acid, phthalic acid, terephthalic acid, tetra and hexahydrophthalic acid, isophthalic acid or trimellitic acid or of partially polymerized, e.g. dimerised, fatty acids.

Exemplary of polyepoxides that differ from glycidyl compounds are the diepoxides of vinylcyclohexene and dicyclopentadiene, 3-(3′,4′-epoxycyclohexyl)-8,9-epoxy-2,4-dioxaspiro[5.5]undecane, the 3′,4′-epoxycyclohexylmethyl ester of 3,4-epoxycyclohexanecarboxylic acid, butadiene diepoxide or isoprene diepoxide, epoxidized linoleic derivatives or epoxidized polybutadiene.

Other suitable epoxy resins are diglycidyl ethers or advanced diglycidyl ethers of dihydric phenols or dihydric aliphatic alcohols of 2 to 4 carbon atoms, preferably the diglycidyl ethers or advanced diglycidyl ethers of 2,2-bis(4-hydroxyphenyl)propane and bis(4-hydroxyphenyl)methane or a mixture of these epoxy resins.

Suitable epoxy resin hardeners for the practice of this invention may be any suitable epoxy resin hardener, typically selected from primary and secondary amines and their adducts, cyanamide, dicyandiamide, polycarboxylic acids, anhydrides of polycarboxylic acids, polyamines, polyamino-amides, polyadducts of amines and polyepoxides and polyols.

A variety of amine compounds (mono, di or polyamines) can be used as a hardener such as aliphatic amines (diethylene triamine, polyoxypropylene triamine etc), cycloaliphatic amines (isophorone diamine, aminoethyl piperazine or diaminocyclohexane etc), or aromatic amines (diamino diphenyl methane, xylene diamine, phenylene diamine etc). Primary and secondary amines broadly can serve as hardening agents while tertiary amines generally act as catalysts.

Although epoxy hardeners are typically amines, other options exist and these will give extra flexibility to accommodate chemical agents that might be unstable or soluble in the presence of amine, or allow a broader range of cure rates to be achieved.

For example, other suitable hardeners are anhydrides of polycarboxylic acids, typically phthalic anhydride, nadic anhydride, methylnadic anhydride, methyltetrahydrophthalic anhydride, methylhexahydrophthalic anhydride and, in addition, tetrahydrophthalic anhydride and hexahydrophthalic anhydride.

For the present invention, certain epoxy polymers are preferred. Preferred epoxy polymers are the polymerized products from one or more preferred epoxy monomers and one or more preferred amine hardeners. Preferred epoxy monomers include: cyclohexanedimethanol diglycidyl ether, neopentyl glycol diglycidyl ether, 1,4-butanediol diglycidyl ether, bisphenol A diglycidyl ether, resorcinol diglycidyl ether, glycerol diglycidyl ether, polyethylene glycol diglycidyl ether, polypropyleneglycol diglycidyl ether, 3,4-epoxycyclohexylmethyl 3,4-epoxycyclohexanecarboxylate, diglycidyl 1,2-cyclohexanedicarboxylate, isosorbide diglycidyl ether, and 1,6-hexanediol diglycidyl ether. Preferred amine hardeners include: Polyoxypropylene diamine, polyoxypropylene triamine, polyoxyethylene diamine, N-aminoethyl-piperazine, trimethyl-1,6-hexanediamine, isophorone diamine, N,N-dimethyl-1,3-diaminopropane, diethylene triamine, N,N′-dimethylethylenediamine, and hexamethylenediamine.

Suitable catalysts such as tertiary amines, borontrifluoride, monoethylamine, imidazoles, triethanolamine, aminoethylpiperazine, tri(dimethylaminomethyl)phenol, bis(dimethylaminomethyl) phenol and dicyandiamides can be optionally used to accelerate the epoxy curing reaction.

### Colloidal Solids

In accordance with the invention, Pickering colloidal emulsion stabilizers of any type may be used to stabilize emulsions prior to the step of solidifying the dispersed phase into a polymer matrix, regardless of polymer matrix type, where the dispersed phase contains a chemical agent such as an agrochemical active ingredient.

More specifically, solids, such as silicas and clays, have been taught in the literature for use as viscosity modifiers in agrochemical formulations to inhibit gravity-driven sedimentation or cream separation by forming a network or gel throughout the continuous phase, thereby increasing the low-shear viscosity, and slowing the movement of small particles, surfactant micelles or emulsion droplets. The colloidal solids of the present invention instead serve to stabilize the droplets containing the resin monomers during cure by adsorbing to the transient liquid-liquid interface, thereby forming a barrier around the curing droplets so that contacting or neighbouring curing droplets are not able to coalesce, irrespective of whether or not the curing droplets have collected in a sediment or a cream layer. The colloidal solids also serve to prevent the GM's from congealing under stress conditions as is observed when plasticizers are imbibed into conventional latex dispersions. It is possible to distinguish the two different functions—rheological modification or emulsion and dispersion stabilization, by a functional test such as described below. The effectiveness of the colloidal solid in stabilizing the emulsions of curing polymer droplets depends on particle size, particle shape, particle concentration, particle wettability and the interactions between particles. The colloidal solids must be small enough so that they can coat the surfaces of the dispersed curing liquid polymer droplets, and the curing liquid droplets must be sufficiently small for use in conventional application equipment. The final polymer particles (and hence, the colloidal solids) will also need to be small enough to provide an acceptably even product distribution at the target site. The colloidal solid also must have sufficient affinity for both the liquids forming the dispersed and continuous phases so that they are able to adsorb to the transient liquid-liquid interface and thereby stabilize the emulsion during cure. This wetting characteristic, particle shape and suitability for Pickering-type emulsion stabilization may be readily assessed by preparing a control formulation lacking the colloidal solid as emulsion stabilizer. In such a case the curing liquid polymer droplets coalesce and form a consolidated mass instead of a dispersion of polymer particles.

In one embodiment, the colloidal solids have a number-weighted median particle size diameter as measured by scanning electron microscopy of 0.001-2.0 microns, particularly 0.5 microns or less, more particularly 0.1 microns or less.

A wide variety of solid materials may be used as colloidal stabilizers for preparing the dispersions of the present invention including carbon black, metal oxides, metal hydroxides, metal carbonates, metal sulfates, polymers, silica, mica and clays.

Suitable colloidal stabilizers are insoluble in any of the liquid phases present in preparation of the concentrate formulation. If an agrochemical active ingredient has suitably low solubility in any liquid used to dilute the final composition, and in both the continuous and (transient) dispersed liquid phases, that is below about 100 ppm at room temperature, and can be prepared at a suitable particle size, and has suitable wetting properties for the transient liquid-liquid interface as described above, then it is also possible that this active ingredient can serve as the colloidal stabilizer. Examples of particulate inorganic materials are oxy compounds of at least one of calcium, magnesium, aluminium and silicon (or derivatives of such materials), such as silica, silicate, marble, clays and talc. Particulate inorganic materials may be either naturally occurring or synthesized in reactors. The particulate inorganic material may be a mineral chosen from, but not limited to, kaolin, bentonite, alumina, limestone, bauxite, gypsum, magnesium carbonate, calcium carbonate (either ground or precipitated), perlite, dolomite, diatomite, huntite, magnesite, boehmite, sepiolite, palygorskite, mica, vermiculite, illite, hydrotalcite, hectorite, halloysite and gibbsite. Further suitable clays (for example aluminosilicates) include those comprising the kaolinite, montmorillonite or illite groups of clay mineral. Other specific examples are attapulgite, laponite and sepiolite. Polymers that flocculate the colloids (such as xanthan in the case of colloidal kaolin) can also improve the stability of Pickering emulsions. Other polymers suitable as colloid solids include cross-linked star polymers such as those exemplified in Saigal et al. [Trishna Saigal, Alex Yoshikawa, Dennis Kloss, Masanari Kato, Patricia Lynn Golas, Krzysztof Matyjaszewski, Robert D. Tilton “Stable emulsions with thermally responsive microstructure and rheology using poly(ethylene oxide) star polymers as emulsifiers”, Journal of Colloid and Interface Science 394 (2013) 284-292].

The type and amount of colloidal solid is selected so as to provide acceptable physical stability of the composition during cure, polymerization, solvent evaporation or other polymer solidification processes. The colloidal solid should also be present in an amount to provide for a stably-dispersed composition. The term “stably-dispersed” as used herein means that under optical microscopy the particles are substantially round spheres (in suspension) and on dilution are visibly identifiable from each other. This can readily be determined by one of skill in the art by routine evaluation of a range of compositions having different amounts of this component. For example, the ability of the colloidal solids to stabilize the composition can be verified by preparing a test sample with the colloidal solid and it can be confirmed that the emulsion of droplets is stable and does not exhibit coalescence. Coalescence is apparent by the formation of large droplets visible to the eye, and ultimately by the formation of a layer of liquid monomers, polymer melt or polymer solution within the formulation. Physical stability of the composition during and after cure, polymerization, solvent evaporation or other polymer solidification is acceptable if no significant coalescence is evident and the GM are present as a dispersion.

For example, in one embodiment the colloidal solids are employed in an amount of from 1 to 80%, particularly from 4 to 50% by weight of the dispersed phase. Mixtures of colloidal solids may be employed.

### Plasticizers

The required mechanical properties of the present invention can be achieved by one or a combination of means. In some embodiments, a plasticizer is used. Plasticizers are relatively small, non-reactive molecules (below 1000 Da) that partially solubilize the polymer molecules to allow movement of segments, thereby conferring flexibility and reducing the rigidity of the overall polymer matrix. Plasticizers are chemically diverse and vary according to the polymer matrix in question, being of necessity miscible with any monomers and the final polymer matrix. Plasticizers may be added to the monomers or polymers prior to formation of the GM, or they may be added to the continuous phase after the polymer matrix particles are formed. In other embodiments, the kind of polymer used for formulation can confer the desired mechanical properties. The selection of polymers with relatively long (more than about 5 bond lengths) segments between sites of potential inter-molecular cross-links, such that these segments have a short persistence length (less than the segment length) and a low tendency to form organized crystal-like domains thereby confer flexibility on the overall polymer matrix. In other embodiments, some or all of the monomers or copolymers used may instead of being multi-functional to allow branching or cross-linking of the polymer matrix, have a lower degree of functionality such that during the curing reaction these monomers reduce the overall cross-link density, thereby producing a polymer matrix microparticle of a hardness between 0.001 MPa and 6 MPa. In the case of cross-linked thermoset epoxy polymer matrices, a preferred means to reduce cross-link density includes mixing mono-glycidyl-ethers with the conventional poly-glycidyl-ethers, and/or mixing one or more mono-primary, mono- or di-secondary amines with the conventional di-, tri- or higher-functional primary amine hardeners. Specific preferred mono-epoxides are butyl glycidyl ether, 2-ethylhexyl glycidyl ether, t-butyl glycidyl ether, phenyl glycidyl ether, o-cresyl glycidyl ether, C12-C14 alkyl glycidyl ether, octylene oxide, allyl glycidyl ether, styrene oxide, pentadecyl phenol glycidyl ether and epoxidized soybean oil.

In certain embodiments of the technology, the inclusion of a specific plasticizer will not be need to obtain the desired hardness of the particle. By way of example, and without limitation, the agrochemical active ingredient itself may have chemical and physical properties which would make the inclusion of a plasticizer unnecessary, or allow the active ingredient itself to function as a plasticizer. Other components of the polymer particle may also cause this same effect/function.

### EXAMPLES

The following examples illustrate further some of the aspects of the invention but are not intended to limit its scope. Where not otherwise specified throughout this specification and claims, percentages are by weight.

### Example 1

### Gel particle Emulsion Formulation Preparation

Load all the ingredients of the oil phase as listed in Table 1 into a beaker, followed by mixing with gentle shear until they form a homogeneous and transparent oil phase. In a separate beaker, load the ingredients of the aqueous phase, followed by homogenizing with high shear mixer. Add the premixed oil phase into the aqueous phase, followed by shearing with UltraTurrax mixer (0.5 inch diameter, 10k rpm) until target particle size (10m) is obtained. Polymerize the monomer emulsion at high temperature (80° C.) for 7 to 15 hrs. A dispersant may be added and the formulation sheared with a sawtooth mixer until it becomes a flowable liquid.

TABLE 1
Tefluthrin gel particle emulsion formulation with a plasticizer

| | | w/w % |
|---|---|---|
| oil phase | Tefluthrin | 18.5 |
| | Aromatic 200 ND | 11.3 |
| | Neopentyl diglycidyl ether | 8.1 |
| | (CAS 17557-23-2) | |
| | Trimethyl hexane diamine | 1.77 |
| | (CAS 25620-58-0) | |
| | Huntsman Jeffamine D400 | 1.77 |
| | Triethanol amine | 0.6 |
| aqueous phase | Tap water | 46.56 |
| | 2% Pregel | 2 |
| | Aerosil MOX80 | 1.4 |
| | Propylene glycol | 4 |
| dispersant | Agnique NSC11 NL | 4 |

### Example 2

### Gel Particle Emulsion Preparation With a Different AI and Different Colloid

Gel emulsions of the present invention may be prepared comprising different AI's and different colloids to stabilize the monomers in an emulsion state during the process which is used to prepare the dispersed phase. As an example, gel emulsions were prepared in accordance with the methods described in Example 1, using the ingredients as listed in Table 2 shown below:

TABLE 2
Fludioxonil gel particle emulsion formulation

| | | w/w % |
|---|---|---|
| oil phase | Fludioxonil | 14.10 |
| | Aromatic 150 | 5.28% |
| | Araldite DY-N | 5.18 |
| | (CAS 17557-23-2) | |
| | Resorcinol diglycidyl ether | 5.18 |
| | (CAS 101-90-6) | |
| | n-octyl amine | 5.19 |
| | Huntsman Jeffamine D230 | 0.27 |
| | (CAS 9046-10-0) | |
| water phase | Tap water | 50.77 |
| | 2% Pregel | 7.56 |
| | Clay RLO 7645 | 6.48 |
| dispersant | Agrimer 60L | 1.00 |

### Example 3

### Gel Particle Emulsions Preparation Without a Plasticizer

Gel emulsions of the present invention may also be prepared without a plasticizer. As an example, gel emulsions were prepared in accordance with the methods described in Example 1, using the ingredients listed in Table 3:

TABLE 3
Fludioxonil gel particle emulsion formulation without a plasticizer

| | | w/w % |
|---|---|---|
| oil phase | Fludioxonil | 14.38 |
| | Araldite DY-N | 6.72 |
| | (CAS 17557-23-2) | |
| | Resorcinol diglycidyl ether | 6.72 |
| | (CAS 101-90-6) | |
| | n-octyl amine | 6.76 |
| | Huntsman Jeffamine D230 | 0.34 |
| | (CAS 9046-10-0) | |
| water phase | Tap water | 50.02 |
| | 2% Pregel | 7.60 |
| | Clay RLO 7645 | 6.45 |
| dispersant | Agrimer 60L | 1.00 |

### Example 4

### Incorporating Multiple AI's in the Polymer Matrix

Gel emulsions of the present invention may incorporate more than one AI. As an example, gel emulsions were prepared similar to the method described in Example 1, using the ingredients as listed in Table 4 shown below:

TABLE 4
Fludioxonil/Sedaxane/Mefenoxam gel
particle emulsions formulation

| | | w/w % |
|---|---|---|
| oil phase | Fludioxonil | 3.7 |
| | Sedaxane | 3.7 |
| | Mefenoxam | 11.0 |
| | Resorcinol diglycidyl ether | 8.7 |
| | (CAS 101-90-6) | |
| | Huntsman Jeffamine D400 | 7.9 |
| | (CAS 9046-10-0) | |
| | Huntsman Jeffamine D2000 | 5.2 |
| | (CAS 9046-10-0) | |
| aqueous phase | Tap water | 45.9 |
| | 2% Pregel | 6.9 |
| | Clay RLO 7645 | 6.0 |
| dispersant | Morwet D425 | 1.0 |

### Example 5

### Improved Crop Safety With Maintained Fungicidal Efficacy of Gel Particle Emulsions

Two benzovindiflupyr formulations were prepared to compare the phytotoxicity (% damage) and fungicidal efficacy (% control) of the two formulations. The first formulation was prepared as an emulsifiable concentrate, and the second as a gel emulsion formulation in accordance with the present technology. The comparative formulations were applied at 4× rate equivalent (1×=13.07 oz emulsifiable concentrate/acre=40.3 g AI/acre) to butter squash plants at approximately three weeks after planting. Phytotoxicity and fungicidal efficacy ratings were taken four days after application. The results of such tests are shown in Table 5. Surprisingly, and unexpectedly, the formulation of the present technology provides for both 0% damage to the plant while maintaining 100% disease control.

TABLE 5

| | Benzovindiflupyr | | | |
|---|---|---|---|---|
| | Emulsifiable | | Benzovindiflupyr | |
| | Concentrate | | Gel Emulsion | |
| Plant # | % Damage | % Control | % Damage | % Control |
| 1 | 22 | 100 | 0 | 100 |
| 2 | 35 | 100 | 0 | 100 |
| 3 | 28 | 100 | 0 | 100 |

### Example 6

### Improved Rainfastness of the Present Technology

Six agrochemical formulations comprising difenoconazole were prepared to compare adhesion properties. A suspension concentrate which includes difenoconazole sold under the tradename Quadris®Top was used for comparative purposes. The six agrochemical formulations were prepared as gel emulsion formulations with increasing particle softness as shown in the table 6b. Hardness was measured using the nanoindenter technique. The gel emulsions were prepared in accordance to table 6a, with varying proportions of neopentyl diglycidyl ether and resorcinol diglycidyl ether to adjust the hardness.

TABLE 6a

| | | w/w % |
|---|---|---|
| oil phase | Difenoconazole | 14.74 |
| | Aromatic 200 | 19.14 |
| | Neopentyl diglycidyl ether (CAS 17557-23-2) | 0-6.8 |
| | Trimethyl hexane diamine (CAS 25620-58-0) | 2.19 |
| | Resorcinol diglycidyl ether (CAS 101-90-6) | 0-6.49 |
| aqueous | Tap water | 39.85 |
| phase | 2% xantham gum gel | 3.35 |
| | Kaolin | 7 |
| | Propylene glycol | 5 |
| dispersant | Agrimer 60 L, 50% | 0.65 |

TABLE 6b

| Formulation | Hardness (MPa) | |
|---|---|---|
| GM 1 | 4.350 | Least Soft |
| GM 2 | 0.194 | [symbol] |
| GM 3 | 0.134 | |
| GM 4 | 0.091 | |
| GM 5 | 0.001 | Softest |
| GM 6 | N/A | Viscous Liquid |

Examples 6a, 6b Quadris®Top and the six gel emulsions were applied to soybean leaves. Rain simulation was applied using the following parameters: a flat fan nozzle (TeeJet 11008 EVS) for large droplet formation, spray intensity: 0.8 g per minute, nozzle height: 20 inches, sprayer speed: 3 mph. Before the simulations, a beaker was placed in the chamber to quantify the rainfall amount. Rainfall simulations were complete after leaves received 1 cm of rainfall. Leaves were then dried for an hour before samples were taken for difenoconazole retention analysis.

### Example 6a

TABLE 7

| | Difenoconazole Retention |
|---|---|
| Formulation | Example 6a |
| Quadris Top (Suspension Concentrate) | 22.07% |
| GM 1 | 33.98% |
| GM 3 | 69.26% |
| GM 4 | 68.45% |
| GM 5 | 73.65% |
| GM 6 | 70.05% |

### Example 6b

TABLE 8

| | Difenoconazole Retention |
|---|---|
| Formulation | Example 6b |
| Quadris Top (Suspension Concentrate) | 25.24% |
| GM 1 | 28.76% |
| GM 2 | 51.58% |
| GM 3 | 57.96% |
| GM 4 | 68.53% |
| GM 5 | 51.04% |
| GM 6 | 40.36% |

Example 6c (average of 6a and 6b):

TABLE 9

| | Difenoconazole Retention |
|---|---|
| Formulation | Example 6c Average |
| Quadris Top (Suspension Concentrate) | 23.66% |
| GM 1 | 31.37% |
| GM 2 | 51.58% |
| GM 3 | 63.61% |
| GM 4 | 68.49% |
| GM 5 | 62.35% |
| GM 6 | 55.21% |

### Example 7

### Comparison Between Feasibility of Imbibing an Organic Liquid Into a Conventional Latex and a Gel Emulsion

The following mixtures, shown in Table 10 were prepared of commercial latex products with the organic hydrophobic liquid herbicide S-metolachlor. The mixtures were designed such that the final compositions would each contain approximately the same amount of polymer.

TABLE 10

| Sample | | | Latex | Water | S-metolachlor |
|---|---|---|---|---|---|
| ID | Product | Description | added [g] | added [g] | added [g] |
| 1.1 | EvoVAE | vinyl | 90.9 | 59.5 | 50 |
| | 401 | acetate/ | | | |
| | | ethylene | | | |
| | | latex | | | |
| 1.2 | EvoVAE | vinyl | 90.9 | 59 | 50 |
| | 405 | acetate/ | | | |
| | | ethylene | | | |
| | | latex | | | |
| 1.3 | Flo Rite | styrene/ | 125 | 25 | 50 |
| | 1197 | butadiene | | | |
| | | latex | | | |
| | | formulated | | | |
| | | with mineral | | | |
| | | fillers | | | |

All 3 samples were left to mix overnight, after which they were low viscosity, uniform latex dispersions. The samples were also all physically unchanged after 4 months at ambient temperature. The Dv50 particle sizes by light scattering were respectively 0.43, 0.48 and 12.4 microns. These observations show that in each case the S-metolachlor can be readily imbibed into the polymer particles that comprise conventional latexes and that the resulting compositions have good dispersion properties at ambient conditions, as has been previously disclosed by other workers.

A GM blank (without active ingredient), ID 1.4, was prepared as follows. 24.1 g of bis-phenol A diglycidiyl ether was mixed with 11.9 g of Jeffamine D-400, ie. a 25% molar excess of bis-phenol A monomer in order to reduce the cross-link density. 32g of this liquid was dispersed with high shear into an aqueous phase comprising 38.8 g water, 3.4 g gel of 2% xanthan in water, 1.3 g of Infilm 939 kaolin Pickering stabilizer and 4.5 g glycerol. The preparation was cured at 50C overnight, 0.4 g of Agrimer 30 dispersant was added and the resulting GM had a Dv50 particle size by light scattering of volume weighted median 208 microns. Although relatively coarse, this sample had excellent stability at ambient conditions and remained a flowable liquid after 4 months.

The GM blank, ID 1.4, was divided into three 10 g aliquots, S-metolachlor was added in the amounts respectively of 38%, 50% and 58% and these aliquots were gently agitated over a weekend. In each case the dispersed phase congealed and formed a single soft rubbery plug that was relatively clear and homogeneous. These observations show that S-metolachlor was imbibed into the epoxy resin of the GM blank and did not remain dispersed in the aqueous phase, but the polymer particles of the GM did not remain dispersed.

This example shows that whereas conventional latex compositions, in which the latex is stabilized by conventional surfactants, can efficiently imbibe an oily liquid and remain dispersed, it is not necessarily possible to imbibe an oily liquid into a GM comprising a cross-linked epoxy resin stabilized by a Pickering colloid at its surface. The reason for the failure of the imbibing process with the GM here is not definitively known and no explanation is offered; this example is presented as evidence that GM and imbibed latex technologies are fundamentally different, the behaviors and advantages of one technology cannot be used to anticipate or predict the behavior of the other.

### Example 8

### Physical Stability of Imbibed Latexes and GM's

A GM was prepared according to the present invention with composition analogous to the GM blank described above in Example 7, ID 1.4, but with the S-metolachlor now combined with the monomers prior to formation of the dispersed phase and cross-linking. 7.2 g of bis-phenol A diglycidiyl ether was mixed with 3.6 g of Jeffamine D-400, ie. a 25% molar excess of bis-phenol A in order to reduce the cross-link density. This monomer mixture was divided into two 4.5 g aliquots. One aliquot was combined with 10.5 g of S-metolachlor and the other was combined with 10.5 g of the solvent Hallcomid M-8-10. 13.3 g of these mixtures was each dispersed at high shear into a mixture of 17 g water, 2 g gel of 2% xanthan in water, and 1 g of Infilm 939 kaolin Pickering stabilizer. The mixtures were each cured overnight at 50° C. resulting in GM ID's 1.5 and 1.6 containing respectively either S-metolachlor or Hallcomid M-8-10. Their respective Dv50 particle sizes by light scattering were 14 and 27 microns. The sample ID 1.5 may be contrasted with the attempt described above to imbibe S-metolachlor into the GM blank sample ID 1.4, which although having substantially the same components present, resulted in no dispersed phase. The contrast demonstrates again the difference between the present invention and known methods involving imbibing into polymer latexes.

The imbibed latex preparations 1.1, 1.2 and 1.3 of Example 7 were compared with the GM preparations 1.5 and 1.6 for their physical stability and performance. The samples were subjected to freeze-thaw on a 24 hour cycle for two months after which they were evaluated for flowability and then rinsed through a 50-mesh wire sieve. Acceptability for agricultural spray application requires that the samples leave substantially no residue on the sieve. The results are shown below in Table 11.

TABLE 11

| Sample | | |
|---|---|---|
| ID | Physical state | 50-mesh sieve residue |
| 1.1 | Congealed to a rubbery | Not tested |
| | plug | |
| 1.2 | Highly viscous | Failed with substantial polymeric residue |
| | dispersion | |
| 1.3 | Flowable dispersion | Failed with substantial polymeric residue |
| 1.5 | Flowable dispersion | None |
| 1.6 | Flowable dispersion | None |

These five samples were also compared for their compatibility with concentrated fertilizer solutions as are commonly used in agriculture. In this test 5 g of each sample was combined in graduated cylinders with 95 mL of fertilizer solution “10-34-0”, these numbers representing the wt % of the elements N, P, K. After being left overnight the number of inversions needed to re-homogenize the mixtures was recorded, and the resulting mixture was then rinsed through a 50-mesh wire sieve. Acceptability requires that the samples leave substantially no residue on the sieve. The results are shown below in Table 12.

TABLE 12

| Sample | Inversion to re-homogenize | |
|---|---|---|
| ID | in 10-34-0 | 50-mesh sieve residue |
| 1.1 | 1 large rubbery plug could | Failed. Impossible to rinse |
| | not be redispersed | through |
| 1.2 | 1 large rubbery plug could | Failed. Impossible to rinse |
| | not be redispersed | through |
| 1.3 | Coarse agglomerates could | Failed. Impossible to rinse |
| | not be redispersed | through |
| 1.5 | 2 | None |
| 1.6 | 8 | None |

These observations show that the imbibed latex preparations, although having initially good dispersion properties and being stable at ambient, have unacceptable physical stability under stress conditions, and are then prone to coalescence and failure of the dispersions so that they can no longer be sprayed as is required of agrochemicals. By contrast the compositions of the present invention have excellent physical stability under a variety of commercially relevant stress conditions.

### Example 9

### Films Formed From Imbibed Latexes, Pickering Emulsions, Hard Polymeric Microparticles and GM

A Pickering emulsion of S-metolachlor in an aqueous solution of glyphosate was prepared as described in example 2 of WO2008/030753 and the sample is designated ID 1.7. A hard polymeric microparticle was prepared and designated ID 1.8 comprising a disperse phase of 21.2 wt % mefenoxam (expressed as a percentage of the entire composition), 12.2 wt % resorcinol diglycidyl ether and 6.6 wt % Jeffamine D-230 dispersed in 47 wt % water, 6 wt % kaolin clay and 7 wt % of 2% xanthan gel. A gel emulsion was prepared according to Example 4 and designated 1.9.

0.5 mL of each of the following samples were placed on pre-weighed plastic microscope slides, allowed to dry overnight at ambient and the weights were recorded.

One set of slides was rinsed in flowing water for 30 s, dried and then weighed. A second set of slides was soaked overnight in water, rinsed, dried and weighed. It should be noted first that samples 1.1 and 1.2 formed extremely sticky clear films, as expected from plasticized film-forming latexes. The stickiness of these films would create severe problems if any dried residues of the imbibed latexes were allowed to form on a plastic surface.

TABLE 11

| Sample ID | Weight loss in 30 s rinse | Weight loss by soaking and rinsing |
|---|---|---|
| 1.1 | 0% | 11% |
| 1.2 | 0% | 8% |
| 1.3 | 0% | 14% |
| 1.7 | 100% | 100% |
| 1.8 | 96% | 94% |
| 1.9 | 18% | 41% |

For samples 1.1, 1.2 and 1.3 the weight loss on soaking is less than the percentage of S-metolachlor present in the compositions, suggesting that while some of the partially water-soluble S-metolachlor has dissolved out of the films, essentially none of the polymeric component has been removed.

These observations show that dried films formed from imbibed conventional latexes are extremely persistent on plastic surfaces to the extent that they effectively cannot be removed. This is not surprising because such latexes are typically used as film formers in paints. Given the prevalence of plastic surfaces in containers and farm equipment, this means that imbibed latexes are impractical to deliver pesticides, and indeed there do not appear to be any commercial uses of latexes for this purpose. Dried deposits would accumulate in sticky films, making the equipment unusable and containing unwanted residues of pesticides.

By contrast the conventional Pickering emulsion, ID 1.7, is extremely easy to redisperse, and indeed this is an advantage of that technology in situations where redispersion is desired. The hard polymeric microparticle, ID 1.8, is also easy to remove from plastic surfaces, even when allowed to form dried films, because the colloid at the surfaces of the polymer particles does not allow the particles to coalesce as long as the particles are rigid.

The GM's of the present invention are quite different from these other technologies. The films comprising GM's of present technology are not sticky to the touch because of the colloid coating on the polymer particles. Their properties can be controlled as taught here to have a desirable intermediate adhesiveness such that there is improved adhesion to surfaces but not to the extent that dried deposits cannot be removed.

### Example 10

### Microcapsules vs Pickering Stabilized Microcapsules

Microcapsules and Pickering Stabilized Microcapsules are generally known in the art. The addition of a Pickering colloid to a microcapsule is generally expected to reduce the adhesiveness of a microparticle to a given surface. Example 10-1 and 10-2 are provided herein to show such a result.

Example 10-1: Polyurea microcapsules of s-Metolachlor with the compositions shown in table 10a were prepared by the following procedure. All the ingredients of the oil phase were loaded into a beaker, followed by mixing with gentle shear until they form a homogeneous and transparent oil phase. In a separate beaker, the ingredients of aqueous phases were loaded, followed by homogenizing with a high shear mixer. Add the premixed oil phase into the aqueous phase, followed by shearing with UltraTurrax high shear mixer (0.5 inch diameter, 10k rpm) until target particle size of the emulsion was obtained. The hardener was added to the emulsion to form a polymer shell wall. The polymerization reaction was performed at room temperature for 14 hrs with gentle agitation. Dispersant was added as necessary.

TABLE 10a
S-metolachlor microcapsules compositions

| | | | Capsule | |
|---|---|---|---|---|
| | Capsule | | Suspension (CS) 2 | |
| | Suspension (CS) 1 | | (Pickering Stabilized) | |
| | Components | wt % | Components | wt % |
| Oil phase | s-Metolachlor | 40 | s-Metolachlor | 40 |
| | Rubinate M | 2.3 | Rubinate M | 2.3 |
| | (polymeric | | | |
| | MDI) | | | |
| Aqueous | Kraftsperse 1251 | 1 | Aerosil OX50 (fumed | 2 |
| phase | (sodium ligno | | silica) | |
| | sulfonate) | | water | 51.4 |
| | Water | 53.3 | | |
| Hardener | Hexamethylene | 3.3 | Hexamethylene | 3.3 |
| | diamine 30% | | diamine 30% | |
| dispersant | | | Kraftsperse 1251 | 1 |

The adhesion and rainfastness of the two microcapsule formulations were conducted on sesame leaves. Diluted formulations (1 g AI/L) were prepared and then sprayed on the leaves. The treated leaves were dried for 2 hours at room temperature, followed by 1 cm of artificial rain (sprayed water equivalent to 1 cm precipitation). The active ingredient (AI) retained on the leaves was extracted and analyzed by HPLC. The data in 10b shows the AI retention pre-rain. The data in Table 10c shows the AI retention after rain, expressed as a percentage of the sprayed AI. In both Tables, the Pickering microcapsule (CS-2) has lower AI retention than the non-Pickering microcapsule (CS-1). This displays that Pickering emulsion system itself does not provide improved stickiness or rainfastness on leaf surfaces.

TABLE 10b
Retention Results for Microcapsules

| | CS-1 | CS-2 |
|---|---|---|
| S-Metolachlor retention | 34.5 | 27.0 |
| ((%) before rain) | | |

TABLE 10c
Rainfastness Results for Microcapsules

| | CS-1 | CS-2 |
|---|---|---|
| S-Metolachlor retention | 3.8 | 1.7 |
| ((%) after rain) | | |

Example 10-2: A tefluthrin polyurea microcapsule, TFT CS-1, was prepared by the following procedure. All the ingredients of the oil phase as listed in Table 10d were load into a beaker, followed by mixing with gentle shear until they formed a homogeneous and transparent oil phase. In a separate beaker, the ingredients of the aqueous phase were loaded, followed by homogenizing with high shear mixer. Add the premixed oil phase into the aqueous phase, followed by shearing with UltraTurrax high shear mixer (0.5 inch diameter, 10 k rpm) until target particle size of the emulsion was obtained. The hardener was added to the emulsion to form a polymer shell wall. The polymerization reaction was performed at room temperature for 14 hrs with gentle agitation.

TABLE 10d
Tefluthrin microcapsule composition

| | TFT CS-1 | | TFT Gel Emulsion | |
|---|---|---|---|---|
| | components | Wt % | components | Wt % |
| Oil phase | Tefluthrin | 14 | Tefluthrin | 18.5 |
| | Shellsol A | 18 | Aromatic 200 ND | 11.3 |
| | Rubinate M | 5 | Neopentyl diglycidyl ether | 8.1 |
| | (polymeric MDI) | | (CAS 17557-23-2) | |
| | | | Trimethyl hexane diamine | 1.77 |
| | | | (CAS 25620-58-0) | |
| | | | Huntsman Jeffamine D400 | 1.77 |
| | | | Triethanol amine | 0.6 |
| Aqueous | Kraftsperse 1251 | 1 | Tap water | 46.56 |
| phase | (sodium ligno | | 2% Pregel | 2 |
| | sulfonate) | | Aerosil MOX80 | 1.4 |
| | Water | 62 | Propylene glycol | 4 |
| Hardener | Hexamethylene | 3.3 | Agnique NSC11 NL | 4 |
| | diamine 30% | | | |

The rainfastness of the microcapsule and the gel emulsion formulations in accordance with the present invention were conducted on glass plates (Table 10e). Diluted formulations (1 g AI/L) were prepared and then sprayed on the glass plates. The treated glass plates were dried for 2 hours at room temperature, followed by 1cm of artificial rain (sprayed water equivalent to 1 cm precipitation). The AI content retained on the glass plates were extracted and analyzed with HPLC.

The data in Table 10e shows the AI retention after rain expressed as a percentage of the AI applied, where the TFT Gel Emulsion shows 2.5 times better rainfastness than the microcapsule (TFT CS-1). The results indicates that the soft property of the GM is beneficial for rainfastness.

TABLE 10e
Rainfastness Results for Microcapsule vs Gel Emulsion

| | TFT CS-1 | TFT Gel Emulsion |
|---|---|---|
| AI (Tefluthrin) retention (%) after rain | 2.0 | 4.9 |

Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims.

1. A liquid dispersion concentrate composition comprising:
(a) a continuous phase; and
(b) at least one dispersed phase comprising a polymer matrix microparticle, wherein the polymer matrix microparticle has: (1) a hardness greater than 0.001 MPa and less than 6 MPa, (2) a colloidal solid material present at the interface with the continuous phase, and (3) an agrochemical active ingredient distributed therein.

2. The composition of claim 1, wherein a colloidal solid material is present in an amount effective to stabilize the polymer matrix microparticles in a stably-dispersed state.

3. (canceled)

4. (Currenty Amended) The composition of claim 2, wherein the composition is free of an emulsifying surfactant.

5. The composition of claim 1, wherein the polymer matrix microparticle further comprises a plasticizer.

6. The composition of claim 1, wherein the polymer matrix microparticle has a hardness greater than 0.001 MPa and less than 5 MPa.

7. The composition of claim 1, wherein the polymer matrix microparticle has a hardness greater than 0.01 MPa and less than 5 MPa.

8. The composition of claim 1, wherein the polymer matrix microparticles are thermoset.

9. The composition of claim 1, wherein the polymer matrix microparticles are thermoplastic.

10. The composition of claim 1, wherein the continuous phase comprises water, one or more substantially water-miscible, non-aqueous liquids, or a mixture of water and one or more water-miscible liquids.

11. The composition of claim 10, wherein the substantially water-miscible, non-aqueous liquid is selected from propylene carbonate, ethylene glycol, diethylene glycol, triethylene glycol, propylene glycol, dipropylene glycol, tripropylene glycol, butylene glycol, hexylene glycol, polyethylene glycols having a molecular weight of up to about 800, di(propylene glycol), diacetin, triacetin, methyl ether acetate, propylene glycol diacetate, triethyl phosphate; ethyl lactate, gamma-butyrolactone, propanol, tetrahydrofurfuryl alcohol, N-methyl pyrrolidone, dimethyl lactamide, and mixtures thereof.

12-13. (canceled)

14. The composition of claim 2, wherein the continuous phase further comprises at least one agrochemically active ingredient and the active ingredient is in the state selected from a solution, an emulsion, a microemulsion, or a suspension of microcapsules or particles.

15. The composition of claim 1, wherein the continuous phase (a) further comprises one or more dispersants.

16. The composition of claim 2, wherein the colloidal solid comprises a particulate inorganic material distributed at the surface of the polymer matrix microparticles.

17. The composition of claim 1, wherein the dispersed phase (b) comprises a cured epoxy resin polymer.

18. The composition of claim 1, wherein the dispersed phase (b) comprises a cured phenolic resin polymer.

19-22. (canceled)

23. The composition of claim 1, wherein the dispersed phase (b) comprises a thermoplastic polystyrene or polyacrylate polymer or biodegradable thermoplastic polymer.

24. The composition of claim 1, wherein the polymer matrix microparticles comprise a combination of polymers selected from a cured epoxy resin polymer, a cured phenolic resin polymer, a cured polyurethane polymer, a cured polyurea resin polymer, a cured aminoplast resin polymer, an unsaturated polyester, a vinyl ester resin polymer, a thermoplastic polystyrene, a polyacrylate polymer, and a biodegradable thermoplastic polymer.

25. The composition of claim 1, wherein (b) comprises a the polymer matrix microparticle is a cured epoxy resin polymer matrix microparticle prepared by curing an epoxy resin selected from mono- di- and polyepoxide monomers, prepolymers, biodegradable epoxy resins or blends thereof with a hardener selected from primary and secondary amines and their adducts, cyanamide, dicyandiamide, polycarboxylic acids, anhydrides of polycarboxylic acids, polyamines, polyamino-amides, polyadducts of amines and polyepoxides, polyols and mixtures thereof.

26. The composition of claim 1, wherein each dispersed phase comprises polymer matrix microparticles with median diameter between 1 and 100 microns.

27-39. (canceled)

40. A polymer matrix microparticle having a hardness greater than 0.001 MPa and less than 6 MPa, a median diameter of between 1 and 100 microns, and comprising at least one entrapped agrochemically active ingredient that is either homogeneously on non-homogeneously distributed within such particle, and wherein the outside surface region of such particle comprises a colloidal solid material.

41. (canceled)
